(19) United States
(12) Patent Application Publication
(10) Pub. No.: US 2023/0263693 A1
(43) Pub. Date: Aug. 24, 2023

(54) MOVEMENT SUPPORT DEVICE AND MOVEMENT SUPPORT SYSTEM

(71) Applicant: TOYOTA JIDOSHA KABUSHIKI KAISHA, Aichi-ken (JP)

(72) Inventors: Kohei SHINTANI, Nisshin-shi (JP); Hiroaki KAWAMURA, Nagoya-shi (JP)

(21) Appl. No.: 18/172,309

(22) Filed: Feb. 22, 2023

(30) Foreign Application Priority Data
Feb. 24, 2022 (JP) 2022-026572

(51) Int. Cl. A61H 3/06

(52) U.S. Cl. CPC A61H 3/061; A61H 3/068; A61H 2201/5064; A61H 2201/5079; A61H 2003/063; A61H 2201/5048; A61H 2201/0153; A61H 2201/5097

(57) ABSTRACT

A movement support device is provided, which early recognize a possibility of contact of a user with a moving body and perform an appropriate movement support operation according to the recognition. It is determined whether there is a possibility of contact of a vehicle with a white cane while maintaining a distance from the vehicle based on a relative position relationship with the vehicle, which is recognized by images from a camera, and a change thereof. When it is determined that there is a possibility of contact, the movement support operation is performed. Thus, the possibility of contact of the vehicle with the user can be early recognized, and when there is a possibility of contact, the movement support operation according to the actual situation can be immediately started, which results in an appropriate acquisition of a start timing of the movement support operation.

### CROSS-REFERENCE TO RELATED APPLICATIONS

The present application claims priority under 35 U.S.C. §119(a) to Japanese Patent Application No. 2022-026572, filed on Feb. 24, 2022. The contents of this application are incorporated herein by reference in its entirety.

### TECHNICAL FIELD

The present invention relates to a movement support device and a movement support system for supporting movement of a user (for example, walking of a visually impaired person using a white cane). In particular, the present invention relates to improvement in processing of information acquired to perform movement support (movement support operation).

### BACKGROUND ART

A movement support device as disclosed in Patent Document 1 is known, which is to support walking of a pedestrian such as a visually impaired person (i.e. to perform movement support for a user using a movement support apparatus). Patent Document 1 discloses a movement support device including: a direction decision unit that determines the walking direction in which a person who behaves without the sense of sight (a visually impaired person) walks; and a guide information generation unit that generates guide information for the visually impaired person to walk in the determined direction. In the disclosure in Patent Document 1, the walking direction in which the visually impaired person walks is determined by matching an image from a camera carried by the visually impaired person and a reference image stored in advance. Then, the visually impaired person is guided to walk the determined walking direction by voice announcement or the like.

### PRIOR ART DOCUMENT

### Patent Document

Patent Document 1: WO 2018/025531

### SUMMARY OF THE INVENTION

### Problem to Be Solved by the Invention

When a user such as a visually impaired person (i.e. a user using a movement support apparatus) walks across a street (for example, walks across a crosswalk), it is required that the movement support apparatus recognizes moving bodies (such as vehicles) in the vicinity of the user based on information (for example, image information) acquired by a built-in camera or the like and thus performs a movement support operation to avoid contact of the user with the moving body. In particular, it is required to early recognize whether there is a possibility of contact of the user with the moving body, and to immediately start the movement support operation in the case where there is a possibility of contact. However, in the field of the conventional movement support devices, no effective proposal has been provided from the viewpoint of early recognition of the possibility of contact of the user with the moving body, and here there is a room for improvement of the movement support device.

The present invention was made in consideration of the above circumstances, an object of which is to provide a movement support device and a movement support system that early recognize whether there is a possibility of contact of a user with a moving body and that perform an appropriate movement support operation according to the recognition.

### Means for Solving the Problem

In order to achieve the above object, the present invention relates to a movement support device capable of performing a movement support operation to support movement of a user using a movement support apparatus in which the movement support device is provided. The movement support device includes a moving body recognizing section, a relative position recognizing section, a contact determining section, and an information transmitting section. The moving body recognizing section recognizes a moving body that exists in the vicinity. The relative position recognizing section recognizes a relative position relationship with the moving body recognized by the moving body recognizing section. The contact determining section determines whether there is a possibility of contact of the moving body with the movement support apparatus in a state in which there is a distance from the moving body based on contact determination support information including at least one of: information on the relative position relationship with the moving body, which is recognized by the relative position recognizing section; and information on a change of the relative position relationship. The information transmitting section outputs instruction information on the movement support operation to perform the movement support operation when the contact determining section determines that there is a possibility of contact of the moving body with the movement support apparatus.

With the above configuration, when a user using the movement support apparatus moves, the contact determining section determines (estimates) whether there is a possibility of contact of a moving body with the movement support apparatus in the state in which there is a distance from the moving body based on contact determination support information including at least one of: information on the relative position relationship with the moving body (i.e. the relative position of the moving body with respect to the movement support device) recognized by the relative position recognizing section; and information on a change of the relative position relationship (i.e. a change of the relative position of the moving body with respect to the movement support device according to movement of the moving body and/or the user). When it is determined that there is a possibility of contact of the moving body with the movement support apparatus, the information transmitting section outputs instruction information on a movement support operation so as to execute the movement support operation. Thus, the movement support operation to avoid the contact of the movement support apparatus (in other words, the user using the movement support apparatus) with the moving body is started. Thus, with this configuration, it is possible to early recognize the possibility of contact of the user with the moving body, and when there is a possibility of contact, the movement support operation according to the actual situation can be immediately started. As a result, it is possible to appropriately obtain a start timing of the movement support operation.

Also, the contact determining section includes: a preliminary estimating part that performs a preliminary estimating operation; and a contact estimating part that performs a contact estimating operation performed subsequently to the preliminary estimating operation. In the preliminary estimating operation performed by the preliminary estimating part, when a plurality of moving bodies is recognized by the moving body recognizing section, the preliminary estimating part solely extracts a moving body estimated to have the possibility of contact among the plurality of moving bodies based on the information including at least one of; the information on the relative position relationship with each of the plurality of moving bodies; and the information on the change of the relative position relationship. Also, in the contact estimating operation performed by the contact estimating part, the contact estimating part determines whether there is a possibility of contact with only the moving body extracted by the preliminary estimating operation based on the contact determination support information in the state in which there is a distance from the extracted moving body.

In the contact estimating operation performed by the contact estimating part in this configuration, the contact estimating part determines whether there is a possibility of contact with only the moving body extracted by the preliminary estimating operation performed by the preliminary estimating part. Thus, it is not necessary to determine the possibility of contact with respect to all the moving bodies recognized by the moving body recognizing section. In other words, no contact estimating operation is required with respect to the moving body that does not have any possibility of contact. Therefore, it is possible to reduce the calculation burden of the contact estimating part (i.e. to effectively use finite resources), which contributes to reduction of time required to determine the possibility of contact with the moving body.

Specifically in the preliminary estimating operation performed by the preliminary estimating part, an extraction condition of the moving body estimated to have the possibility of contact includes a moving direction of the moving body as a direction in which the moving body approaches the movement support apparatus.

In the preliminary estimating operation in this configuration, the moving body that approaches the movement support apparatus (i.e. that approaches the user) is extracted as a moving body estimated to have a possibility of contact. Thus, it is possible to narrow down the moving bodies with respect to which the possibility of contact is estimated in the contact estimating operation. In other words, the moving body that does not approach the movement support apparatus (i.e. that does not approach the user) is eliminated from the target moving bodies with respect to which the possibility of contact is estimated in the contact estimating operation. Therefore, it is possible to reduce the calculation burden of the contact estimating part.

Specifically in the preliminary estimating operation in this configuration, the preliminary estimating part extracts the moving body estimated to have the possibility of contact according to a moving velocity of the moving body whose moving direction is the direction in which the moving body approaches the movement support apparatus. As to the extraction condition of the moving body estimated to have the possibility of contact, the range of a moving velocity condition for estimating that the moving body has the possibility of contact when the moving body approaches the movement support apparatus without changing the moving direction is set higher than the range of the moving velocity condition for estimating that the moving body has the possibility of contact when the moving body approaches the movement support apparatus with changing the moving direction.

For example, among the vehicles (moving bodies) that enter the intersection, the vehicle that turns right or turns left (i.e. that changes its moving direction) generally has a relatively low velocity, while the vehicle that goes straight ahead (i.e. that does not change its moving direction) generally has a relatively high velocity. Taking into account the above, as to the extraction condition (condition of moving velocity) of the moving body estimated to have the possibility of contact in this configuration, the range of the moving velocity condition for estimating that the moving body has the possibility of contact when the moving body approaches the movement support apparatus without changing the moving direction is set higher than the range of the moving velocity condition for estimating that the moving body has the possibility of contact when the moving body approaches the movement support apparatus with changing the moving direction. Thus, the extraction condition of the moving body estimated to have the possibility of contact is set in light of the actual state of the moving velocity of the moving body. In this way, it is possible to improve extraction reliability of the vehicle estimated to have a possibility of contact.

Also in the preliminary estimating operation, the preliminary estimating part extracts the moving body estimated to have the possibility of contact according to a moving acceleration of the moving body whose moving direction is the direction in which the moving body approaches the movement support apparatus. As to the extraction condition of the moving body estimated to have the possibility of contact, the range of the moving acceleration condition for estimating that the moving body has the possibility of contact when the moving body approaches the movement support apparatus without changing the moving direction may be set higher than the range of the moving acceleration condition for estimating that the moving body has the possibility of contact when the moving body approaches the movement support apparatus with changing the moving direction.

For example, among the vehicles (moving bodies) that enter the intersection, the vehicle that turns right or turns left (i.e. that changes its moving direction) generally has a relatively low acceleration (e.g. the vehicle changes its moving direction with decelerating), while the vehicle that goes straight ahead (i.e. that does not change its moving direction) generally has a relatively high acceleration (e.g. the vehicle moves at the constant velocity or moves while accelerating). Taking into account the above in this configuration also, as to the extraction condition (condition of moving acceleration) of the moving body estimated to have the possibility of contact, the range of the moving acceleration condition for estimating that the moving body has the possibility of contact when the moving body approaches the movement support apparatus without changing the moving direction is set higher than the range of the moving acceleration condition for estimating that the moving body has the possibility of contact when the moving body approaches the movement support apparatus with changing the moving direction. Thus, the extraction condition of the moving body estimated to have the possibility of contact is set in light of the actual state of the moving acceleration of the moving body. In this way also, it is possible to improve extraction reliability of the vehicle estimated to have a possibility of contact.

Also in the preliminary estimating operation by the preliminary estimating part, the extraction condition of the moving body estimated to have the possibility of contact includes a state in which the moving body is being stopped at a position toward a front of the user in a user's moving direction.

When the moving body is being stopped at the position toward the front of the user in the user's moving direction, if the user continues to move, the user may make contact with the moving body. For the above reason, in this configuration, the extraction condition of the moving body estimated to have the possibility of contact includes the state in which the moving body is being stopped at the position toward the front of the user in the user's moving direction. Thus, the moving body is extracted in the preliminary estimating operation, and it is determined whether there is a possibility of contact with this moving body in the contact estimating operation. In this way, it is also possible to early determine whether there is a possibility of contact with the stopped moving body.

Also in the contact estimating operation performed by the contact estimating part, the contact determination support information includes a relative distance between a fixed object located toward the front of the user in the user's moving direction and the moving body.

When the relative distance between the moving body and the fixed object (e.g. the white line of the crosswalk) that is located toward the front of the user in the user's moving direction is relatively large, the moving body has not yet reached in front of the user in the user's moving direction. In this case, the moving body can be determined to have no possibility of contact. On the other hand, when the relative distance between the moving body and the fixed object located toward the front of the user in the user's moving direction is not more than a predetermined value (e.g. not more than 0), the moving body may have reached in front of the user in the user's moving direction. In this case, the moving body can be determined to have a possibility of contact.

Thus, it is possible to estimate whether there is a possibility of contact with the moving body with high accuracy in the contact estimating operation.

Also in the contact estimating operation performed by the contact estimating part, the contact determination support information may include a moving velocity of the moving body.

In the case where the moving velocity of the moving body is high, even when the moving body has not yet reached in front of the user in the user's moving direction at the current time, the moving body may reach in front of the user in the user's moving direction within a relatively short time, which may cause the contact of the user with the moving body. Taking into account the above, in this configuration, the contact determination support information includes the moving velocity of the moving body. Thus, it is possible to determine whether there is a possibility of contact with the moving body with high accuracy in consideration of the moving velocity.

Also in the contact estimating operation performed by the contact estimating part, the contact determination support information may include a relative distance between the moving body and the movement support apparatus.

In the case where the relative distance between the moving body and the movement support apparatus is small, even when the moving body has not yet reached in front of the user in the user's moving direction at the current time, after that the moving body may make contact with the user because the movement of the moving body and the movement of the user continue. Taking into account the above, in this configuration, the contact determination support information includes the relative distance between the moving body and the movement support apparatus. Thus, it is possible to determine whether there is a possibility of contact with the moving body with high accuracy in consideration of the relative distance.

Also, the contact determining section performs a determination operation on whether there is a possibility of contact with the moving body on a condition that the user is crossing a road on which the moving body moves.

The contact of the movement support apparatus with the moving body occurs when the user using the movement support apparatus is crossing the road on which the moving body moves. Thus, since the determination operation on whether there is a possibility of contact with the moving body is performed only when the user is crossing the road on which the moving body moves, performing useless determination operations (for example, the determination operation performed in the situation where the user is moving on the area where no moving body passes) can be reduced.

Also, the movement support device further includes a notifier for the movement support operation, and the notifier gives a notice to the user by vibration or by voice for supporting the movement of the user.

Thus, it is possible to appropriately give a notice to the user using the movement support apparatus.

Also, when the user is assumed to be a visually impaired person and the movement support apparatus is assumed to be a white cane used by the visually impaired person, the movement support device is built in the white cane. Thus, it is possible to provide a highly useful white cane.

In order to achieve the above object, the present invention may also be configured as a movement support system including a movement support device. Specifically, the present invention relates to the movement support system including the movement support device capable of performing a movement support operation to support movement of a user using a movement support apparatus in which the movement support device is provided. The system further includes an instruction information receiving section mounted on a moving body. The movement support device includes: a moving body recognizing section recognizing the moving body that exists in the vicinity; a relative position recognizing section recognizing a relative position relationship with the moving body recognized by the moving body recognizing section; a contact determining section determining whether there is a possibility of contact of the moving body with the movement support apparatus in a state in which there is a distance from the moving body based on contact determination support information including at least one of: information on the relative position relationship with the moving body, which is recognized by the relative position recognizing section; and information on a change of the relative position relationship; and an information transmitting section outputting, to the instruction information receiving section of the moving body, instruction information on the movement support operation to perform the movement support operation when the contact determining section determines that there is a possibility of contact of the moving body with the movement support apparatus. The moving body includes a contact avoidance control section that performs a contact avoiding operation to avoid the contact with the user when the instruction information receiving section receives the instruction information on the movement support operation.

With the above configuration, when it is determined that there is a possibility of contact of the moving body with the movement support apparatus, the information transmitting section transmits the instruction information on the movement support operation to the instruction information receiving section of the moving body. Then, the moving body that received the instruction information on the movement support operation performs the contact avoiding operation by the contact avoidance control section. Examples of the contact avoiding operation include: emitting the voice to call the driver's attention; and generating the braking force to the moving body.

### Effects of the Invention

In the present invention, it is determined whether there is a possibility of contact of a moving body with a movement support apparatus in a state in which there is a distance from the moving body based on contact determination support information including at least one of: information on a relative position relationship with the recognized moving body; and information on a change of the relative position relationship. When it is determined that there is a possibility of contact, instruction information on a movement support operation is output so as to execute the movement support operation. Thus, it is possible to early recognize the possibility of contact of the user with the moving body, and when there is a possibility of contact, the movement support operation according to the actual situation can be immediately started. As a result, it is possible to appropriately obtain a start timing of the movement support operation.

### BRIEF DRAWINGS OF THE INVENTION

FIG. 1 is a diagram illustrating a white cane including a built-in type movement support device according to an embodiment.

FIG. 2 is a schematic diagram illustrating an inside of a handgrip part of the white cane.

FIG. 3 is a block diagram indicating a schematic configuration of a control system of the movement support device.

FIG. 4 is a diagram exemplarily illustrating an image taken by a camera.

FIG. 5 is a front view of a vehicle for explaining vehicle orientation threshold values that define the orientation of the vehicle.

FIG. 6 is a diagram exemplarily illustrating a state in which a user is walking across a crosswalk.

FIG. 7 is a diagram exemplarily indicating a state at a time t−1 in vehicle velocity detecting processing.

FIG. 8 is a diagram exemplarily indicating a state at a time t in the vehicle velocity detecting processing.

FIG. 9 is a diagram for explaining a calculation principle of the vehicle velocity in the vehicle velocity detecting processing.

FIG. 10 is a table for explaining a preliminary estimating operation.

FIG. 11 is a diagram for explaining a contact estimating operation.

FIG. 12 is a diagram exemplarily illustrating an image taken by the camera when the user is in a walking state toward the crosswalk.

FIG. 13 is a diagram exemplarily illustrating an image taken by the camera at a timing when the user arrives at the crosswalk.

FIG. 14 is a diagram exemplarily illustrating an image taken by the camera when the user is in a crossing state on the crosswalk.

FIG. 15 is a diagram exemplarily illustrating an image taken by the camera when the user in the crossing state on the crosswalk is walking in a direction deviating from the crosswalk toward the right direction.

FIG. 16 is a diagram exemplarily illustrating an image taken by the camera when the user in the crossing state on the crosswalk is walking in a direction deviating from the crosswalk toward the left direction.

FIG. 17 is a diagram illustrating an image of a crosswalk and a traffic light that are recognized.

FIG. 18 is a diagram for indicating respective sizes with respect to a boundary box of a white line in the recognized crosswalk.

FIG. 19 is a flowchart indicating a procedure of a walking support operation by the movement support device.

FIG. 20 is a flowchart indicating a procedure of a walking support operation based on vehicle contact estimation by the movement support device.

FIG. 21 is a block diagram indicating a schematic configuration of the control system of the movement support system according to a variation.

FIG. 22 is a diagram corresponding to FIG. 6 for explaining a user's position notification operation according to a variation.

### MODE FOR CARRYING OUT THE INVENTION

Hereinafter, an embodiment of the present invention will be described with reference to the drawings. In this embodiment, the description will be given on the case where a movement support device of the present invention is built in a white cane (movement support apparatus) used by a visually impaired person. Also hereinafter, the visually impaired person is in some cases simply called as a “user”. However, the user in the present invention is not limited to the visually impaired person.

### —Overall Configuration of White Cane—

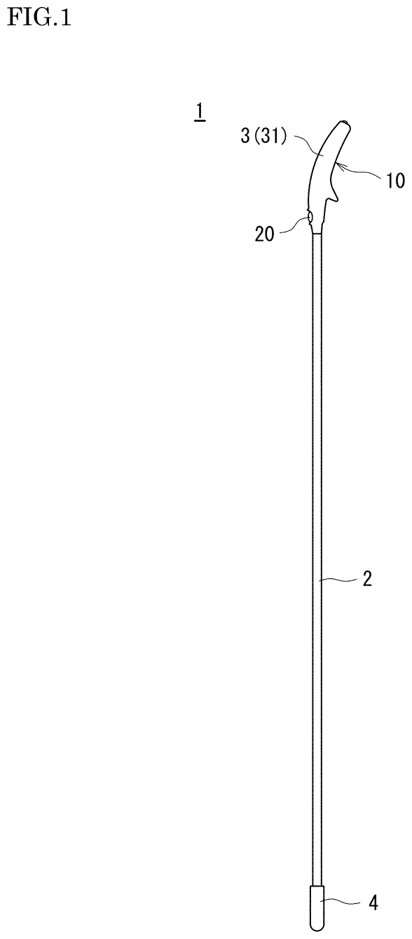

FIG. 1 is a diagram illustrating a white cane 1 in which a movement support device of this embodiment is built. As shown in FIG. 1, the white cane 1 includes: a shaft part 2; a handgrip part 3; and a tip part (shoe) 4.

The shaft part 2 has a hollow rod shape having a substantially circular cross-section. The shaft part 2 is made of aluminum alloy, glass fiber reinforced resin, carbon fiber reinforced resin, or the like.

The handgrip part 3 is provided on a base end part (upper end part) of the shaft part 2, and a cover 31 made of an elastic body such as rubber is attached to the handgrip part 3. Also, the handgrip part 3 of the white cane 1 according to this embodiment has a shape that the top part thereof (upper side in FIG. 1) is slightly bent taking into account ease in holding and non-slipperiness when the user grasps the handgrip part 3. However, the shape of the handgrip part 3 is not limited thereto.

The tip part 4 is a member made of rigid synthetic resin or the like and has a bottomed cylindrical shape. The tip part 4 is fitted and fixed onto the end part of the shaft part 2 by means of adhering or screwing. Also, the end part of the tip part 4 has a hemispherical end surface.

The white cane 1 according to this embodiment is a rigid cane that is not foldable. However, it may be a foldable or extendable type by folding/extending a part or multiple parts in the middle of the shaft part 2.

### —Configuration of Movement Support Device—

The characteristic feature of this embodiment derives from the movement support device 10 that is built in the white cane 1. This movement support device 10 is described here.

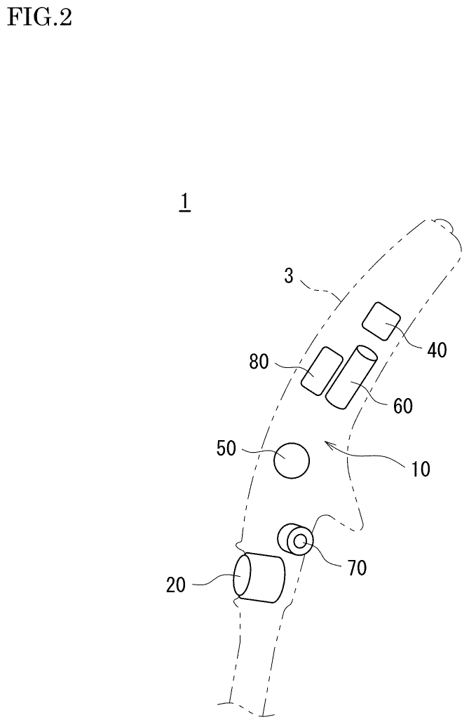

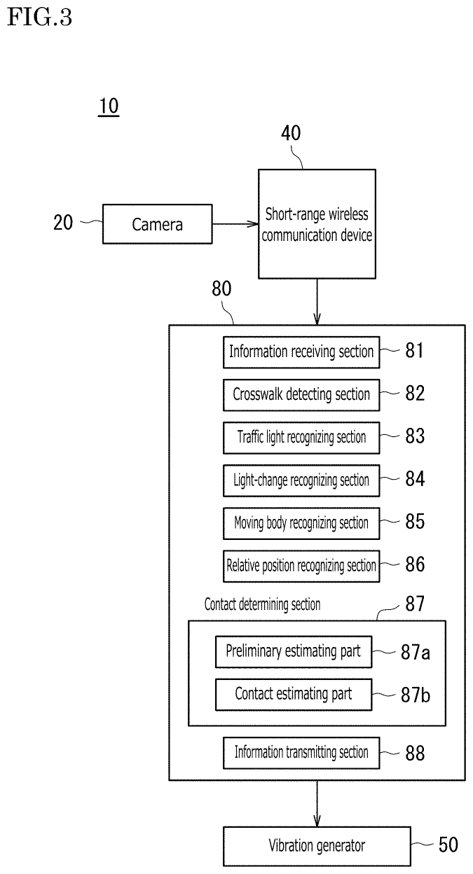

FIG. 2 is a schematic diagram illustrating an inside of the handgrip part 3 of the white cane 1. As shown in FIG. 2, the movement support device 10 according to this embodiment is built in the white cane 1. Also, FIG. 3 is a block diagram indicating a schematic configuration of a control system of the movement support device 10.

As shown in FIGS. 2 and 3, the movement support device 10 includes: a camera 20; a short-range wireless communication device 40; a vibration generator (notifier) 50; a battery 60; a charging socket 70; and a control device 80.

The camera 20 is embedded in the front surface (i.e. the surface facing the traveling direction of the user) of the base of the handgrip part 3 so as to take images in front of the user in the traveling direction (the front side in the walking direction). The camera 20 is, for example, a CCD (Charge Coupled Device) camera or a CMOS (Complementary Metal Oxide Semiconductor) camera. The configuration and/or the provided position of the camera 20 are/is not limited to those described above. As an example, the camera 20 may be embedded in the front surface (i.e. the surface facing the traveling direction of the user) of the shaft part 2.

The camera 20 is characteristically configured as a wide angle camera that can take, as an image in front of the walking user in the traveling direction, an image including both of the following: a white line among the white lines constituting a crosswalk, which is the closest one to the user when he/she arrives at the crosswalk; and a traffic light (for example, a pedestrian traffic light) that is located in front of the user. That is, when the user reaches in front of the crosswalk, the camera can take the image of the closest white line of the crosswalk located in the vicinity of the feet of the user (specifically, located slightly in front of the user's feet) and the traffic light located at the position across the crosswalk. The viewing angle (vertical viewing angle) required for the camera 20 is appropriately set, as described above, so as to be capable of acquiring (taking) images that include both of the white line (crosswalk's white line) located at the closest position to the user and the traffic light. Also, the horizontal viewing angle of the camera 20 is set so as to be capable of taking images of vehicles and the like located on the lateral side of the user. Also, it is preferable that the viewing angle is set so as to be capable of taking images of the vehicles and the like located diagonally backward of the user.

The short-range wireless communication device 40 is a wireless communication device to perform short-range wireless communication between the camera 20 and the control device 80. For example, the short-range wireless communication is performed between the camera 20 and the control device 80 by means of communication such as well-known Bluetooth (registered trademark) so as to wirelessly transmit information on the image taken by the camera 20 to the control device 80.

The vibration generator 50 is provided in the base part of the handgrip part 3, above the camera 20. The vibration generator 50 vibrates by operations of a built-in motor, and transmits the vibration to the handgrip part 3 so as to notify the user who holds the handgrip part 3 of various kinds of information. Specific examples of notification by the vibration of the vibration generator 50 to the user will be described later.

The battery 60 is constituted of a secondary battery that stores electricity for the camera 20, the short-range wireless communication device 40, the vibration generator 50 and the control device 80.

The charging socket 70 is a part to which a charging cable is connected when storing electricity in the battery 60. For example, the charging cable is connected thereto when the user is at home and wants to charge the battery 60 from the domestic power supply.

The control device 80 includes, for example: a processor such as a CPU (Central Processing Unit); a ROM (Read-Only Memory) to store control programs; a RAM (Random-Access Memory) to temporarily store data; and an input/output port.

The control device 80 includes, as function sections to be executed by the control programs: an information receiving section 81; a crosswalk detecting section 82; a traffic light recognizing section 83; a light-change recognizing section 84; a moving body recognizing section 85; a relative position recognizing section 86; a contact determining section 87; and an information transmitting section 88. Hereinafter, respective functions of the above sections are briefly described.

The information receiving section 81 receives, at a predetermined time interval, information on the image taken by the camera 20 from the camera 20 via the short-range wireless communication device 40.

The crosswalk detecting section 82 recognizes the crosswalk and detects the position of each white line of the crosswalk in the image received by the information receiving section 81 as the information on the image (i.e. the information on the image taken by the camera 20).

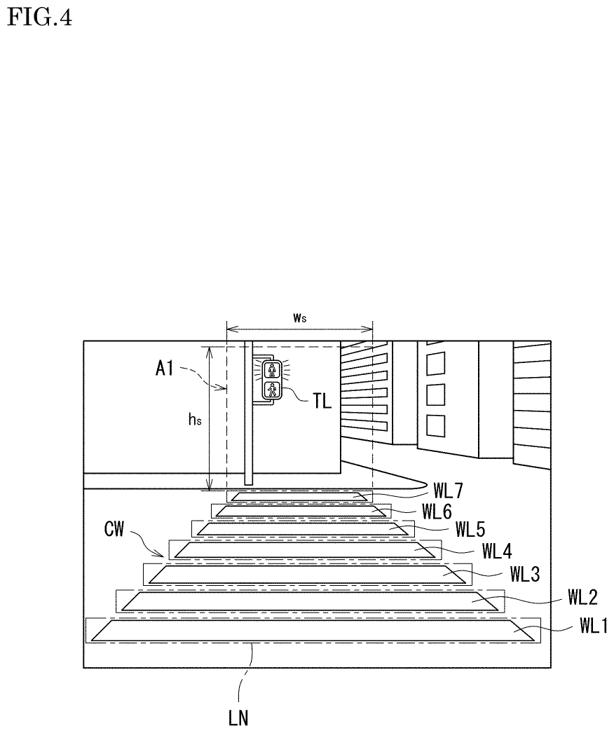

Specifically, as shown in FIG. 4 (one example of the images taken by the camera 20), a boundary box (see the long dashed short dashed lines in FIG. 4) is set with respect to each of a plurality of white lines WL1 to WL7 constituting a crosswalk CW. For example, the white lines WL1 to WL7 of the crosswalk CW are identified by the well-known matching processing, and the boundary box is set with respect to each of the identified white lines WL1 to WL7. Alternatively, the white lines WL1 to WL7 may be identified using annotated data on the white lines (labeled data on the white lines; training data for recognizing the white lines by deep learning) so as to set the boundary box with respect to each of the identified white lines WL1 to WL7.

Then, the crosswalk detecting section 82 detects a lower end position of the boundary box (see the position LN in FIG. 4) located at the position closest to a pedestrian among the above boundary boxes. In this embodiment, the boundary boxes are respectively set with respect to the white lines WL1 to WL7 so as to determine the lower end position LN of the boundary box located at the lowest position in the image to be the “crosswalk's end edge position that is closest to the pedestrian”. However, the “crosswalk's end edge position that is closest to the pedestrian” may be determined to be the lower end position of the white line WL1 located at the lowest position among the plurality of identified white lines WL1 to WL7 in the image. In this case, setting the boundary boxes is not required.

As described later, the boundary boxes are used to: detect the stopping position of the user; detect the position of a traffic light TL; detect the traveling direction when the user walks across the crosswalk CW; determine whether the user finishes crossing the crosswalk CW; detect the position of a vehicle; and calculate the velocity and/or the acceleration of the vehicle. The detail description will be given later.

The traffic light recognizing section 83 determines whether the state of the traffic light TL is red (stop instruction state) or green (crossing permission state) based on information on the image received by the information receiving section 81. When estimating the existing area of the traffic light TL in the image received by the information receiving section 81, in-image coordinates of the boundary box located at the farthest position are identified among the boundary boxes set with respect to the recognized white lines WL1 to WL7 as described above, and furthermore the rectangle that comes into contact with the upper side of the above-described boundary box (i.e. the boundary box set with respect to the white line WL7 located at the farthest position among the recognized white lines WL1 to WL7) is defined, as shown in FIG. 4. Thus, the rectangle having the width of wₛ and the height of hₛ is defined as an area A in which the traffic light TL exists (existing area of the traffic light TL), which is output as the cropped area. This cropped area may be a square or may be a rectangle. In order to detect the state of the traffic light (color detection) by the traffic light recognizing section 83, general algorithms such as an object detection algorithm and a rule-based algorithm are used.

The light-change recognizing section 84 recognizes that the state of the traffic light TL determined by the traffic light recognizing section 83 turns from red to green. When the change of the traffic light is recognized, the light-change recognizing section 84 transmits a light change signal to the information transmitting section 88. The light change signal is further transmitted from the information transmitting section 88 to the vibration generator 50. The vibration generator 50 vibrates in a predetermined pattern in association with receipt of the light change signal so as to notify the user of a permission of crossing the crosswalk (crossing start notification) derived from the change of the traffic light TL from red to green.

The moving body recognizing section 85 recognizes existence of a vehicle (a moving body that exists in the vicinity in the present invention) in the image received by the information receiving section 81 as the information on the image (i.e. the information on the image taken by the camera 20).

Specifically, the moving body recognizing section 85 is a function section to perform a vehicle recognition operation to the image acquired (taken) by the camera 20 using a learned model based on annotated data. More specifically, the moving body recognizing section 85 performs the vehicle recognition operation using deep learning. In other word, by using the annotated data on the vehicle (labeled data on the vehicle; training data for recognizing the vehicle by deep learning), the moving body recognizing section 85 determines whether any vehicle exists or not in the image acquired by the camera 20 (determination on whether any vehicle exists or not), and also recognizes the state of the vehicle (the facing direction of the vehicle, etc.). Examples of the annotated data includes data on vehicle images such as: a front image; a rear image; a right-side image; a left-side image; a front image viewed from the diagonally right direction; a rear image viewed from the diagonally right direction; a front image viewed from the diagonally left direction; and a rear image viewed from the diagonally left direction. That is, various images of the vehicle in the circumferential direction around the vertical axis are annotated as data in advance. Since there are various kinds of vehicles, it is preferable that data corresponding to such various kinds of vehicles (for example, sedan cars, wagon cars and minivans) is annotated in advance.

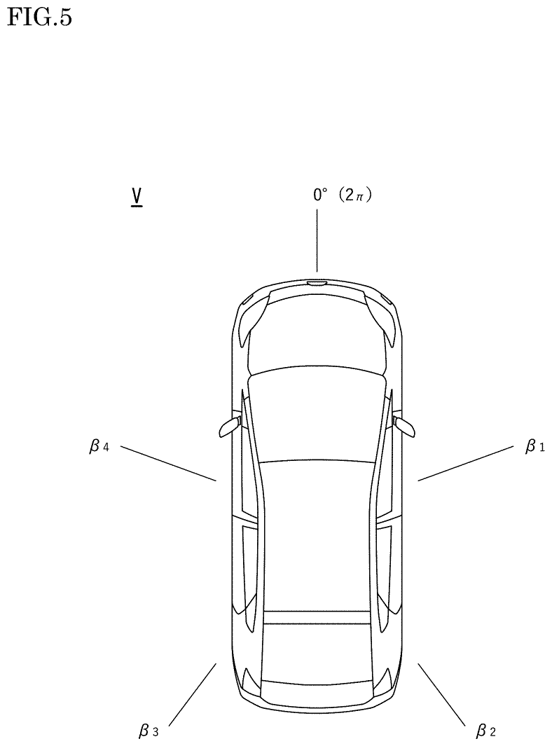

Also in this embodiment, values respectively corresponding to five directions are set in the circumferential direction around the vertical axis of the vehicle. These values are vehicle orientation threshold values to define the vehicle orientation (i.e. the vehicle orientation threshold values used for recognizing which direction the vehicle is facing). Specifically, as shown in FIG. 5, the vehicle orientation threshold value when a vehicle V is viewed from the front is defined as 0° (=2π), and each direction that forms 72° with respect to the adjacent direction in a clockwise in FIG. 5 is defined. Thus, these defined directions are represented by the vehicle orientation threshold values β1, β2, β3 and β4. In this way, in the vehicle V in the image acquired by the camera 20, when the surface facing the camera 20 falls into the vehicle orientation threshold value range of 0° to β1, it is determined that the surface of the vehicle V facing the camera 20 is in the range of the front surface to the right front surface. When the surface facing the camera 20 falls into the vehicle orientation threshold value range of β1 to β2, it is determined that the surface of the vehicle V facing the camera 20 is in the range of the right side surface to the right rear surface. When the surface facing the camera 20 falls into the vehicle orientation threshold value range of β2 to β3, it is determined that the surface of the vehicle V facing the camera 20 is the rear surface. When the surface facing the camera 20 falls into the vehicle orientation threshold value range of β3 to β4, it is determined that the surface of the vehicle V facing the camera 20 is in the range of the left side surface to the left rear surface. When the surface facing the camera 20 falls into the vehicle orientation threshold value range of β4 to 2π, it is determined that the surface of the vehicle V facing the camera 20 is in the range of the front surface to the left front surface.

The relative position recognizing section 86 recognizes the relative position relationship with the vehicle V recognized by the moving body recognizing section 85 (i.e. recognizes the relative position of the vehicle V with respect to the movement support device 10). That is, the relative position recognizing section 86 recognizes the direction in which the vehicle V exists when the vehicle V exists in the image acquired by the camera 20 (in other words, when the existence of the vehicle V is recognized by the moving body recognizing section 85).

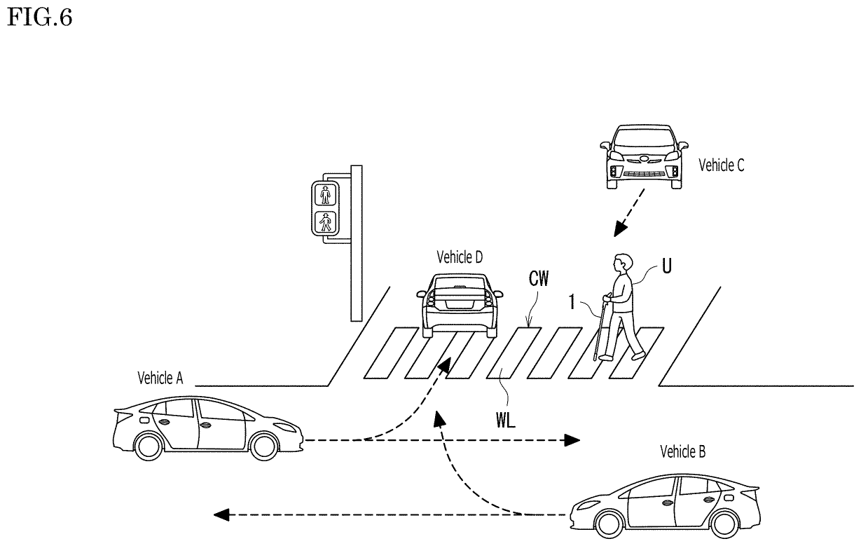

FIG. 6 is a diagram exemplarily illustrating a state in which a user U is walking across the crosswalk CW. FIG. 6 shows the state in which four vehicles A to D are travelling or are stopped (stand by) in the vicinity of an intersection. The vehicle A in FIG. 6 is a vehicle that enters the intersection from the left and diagonally forward direction when viewed from the user U. In this case, the relative position recognizing section 86 recognizes the vehicle A as a vehicle that exists in the left and diagonally forward direction (in particular, it is recognized as a vehicle that exists in the left and diagonally forward direction because of the vehicle position located at the upper left part of the image). The traveling direction of the vehicle A that enters the intersection is estimated to be the straight ahead direction or the left-turn direction as shown by the dashed arrow in FIG. 6 (here, the right-turn and the backward movement are out of consideration). The vehicle B in FIG. 6 is a vehicle that enters the intersection from the left and diagonally backward direction when viewed from the user U. In this case, the relative position recognizing section 86 recognizes the vehicle B as a vehicle that exists in the left and diagonally backward direction (in particular, it is recognized as a vehicle that exists in the left and diagonally backward direction because of the vehicle position located at the lower left part of the image). The traveling direction of the vehicle B that enters the intersection is estimated to be the straight ahead direction or the right-turn direction as shown by the dashed arrow in FIG. 6 (here, the left-turn and the backward movement are out of consideration). The vehicle C in FIG. 6 is a vehicle that enters the intersection from the right direction when viewed from the user U. In this case, the relative position recognizing section 86 recognizes the vehicle C as a vehicle that exists in the right direction (in particular, it is recognized as a vehicle that exists in the right direction because of the vehicle position located on the right side of the image). The traveling direction of the vehicle C that comes toward the intersection (toward the user U) is estimated to be the straight ahead direction, or the vehicle C is estimated to be stopped. The vehicle D in FIG. 6 is a vehicle that is being stopped at a point in the right and diagonally forward direction when viewed from the user U. In this case, the relative position recognizing section 86 recognizes the vehicle D as a vehicle that exists in the right and diagonally forward direction (in particular, it is recognized as a vehicle that exists in the right and diagonally forward direction because of the vehicle position located at the upper right part of the image).

The contact determining section 87 determines whether there is a possibility of contact of the user U with any of the vehicles A to D based on contact determination support information including: information on the relative position relationship with each of the vehicles A to D, which is recognized by the relative position recognizing section 86; and information on a change of the relative position relationship. This determination operation is performed on the condition that the user U is crossing the crosswalk CW taking into account the fact that the contact of the user U with the vehicle occurs when the user U using the white cane 1 is crossing the crosswalk CW. In other words, since the determination operation on whether there is a possibility of contact with the vehicle is performed only when the user U is crossing the crosswalk, performing useless determination operations (for example, the determination operation performed in the situation where the user U is walking on the area where no vehicle passes (e.g. a sidewalk)) can be reduced. Hereinafter, the detail description will be given.

The contact determining section 87 includes: a preliminary estimating part 87a that performs a preliminary estimating operation (described later); and a contact estimating part 87b that performs a contact estimating operation (described later) after the preliminary estimating operation is performed.

### (Preliminary Estimating Operation)

The preliminary estimating operation performed by the preliminary estimating part 87a is to extract solely a vehicle that is estimated to have a possibility of contact with the user U among the plurality of vehicles A to D based on information on the relative position relationship with each of the vehicles A to D and information on a change of the relative position relationship, when the moving body recognizing section 85 recognizes the plurality of vehicles A to D.

More specifically, the preliminary estimating part 87a determines the traveling state of each of the vehicles A to D based on the information on the images transmitted from the camera 20 at predetermined intervals, and thus extracts solely the vehicle that is estimated to have a possibility of contact with the user U among the plurality of vehicles A to D. The traveling state of the vehicle here is an index including the vehicle orientation and the vehicle velocity. The vehicle orientation can be obtained by inference results using the deep learning model (the learned model described above). That is, the vehicle orientation can be determined depending on which range divided by the vehicle orientation threshold values the vehicle orientation belongs to, as described above. Also, the vehicle velocity can be calculated using the amount of vehicle movement per unit time based on the information on the images transmitted from the camera 20 at predetermined intervals.

Here, arithmetic processing of the vehicle velocity is specifically described.

The user walks while swinging the white cane 1 side to side in order to confirm the road surface condition in front of the user. Thus, by swinging the white cane 1 side to side, an imaging optical axis of the camera 20 built in the white cane 1 is also swings side to side. Therefore, the direction of the imaging optical axis of the camera 20 considerably varies with respect to the walking direction of the user. As a result, when a vehicle exists in the image transmitted from the camera 20, it is difficult to correctly calculate the amount of vehicle movement per unit time due to the variations in the direction of the imaging optical axis. In consideration of the above circumstances, in this embodiment, the vehicle velocity is calculated by calculating the velocity vector by the deep learning model using the image taken at the time t and the image taken at the time t−1 that is 1 frame before the time t.

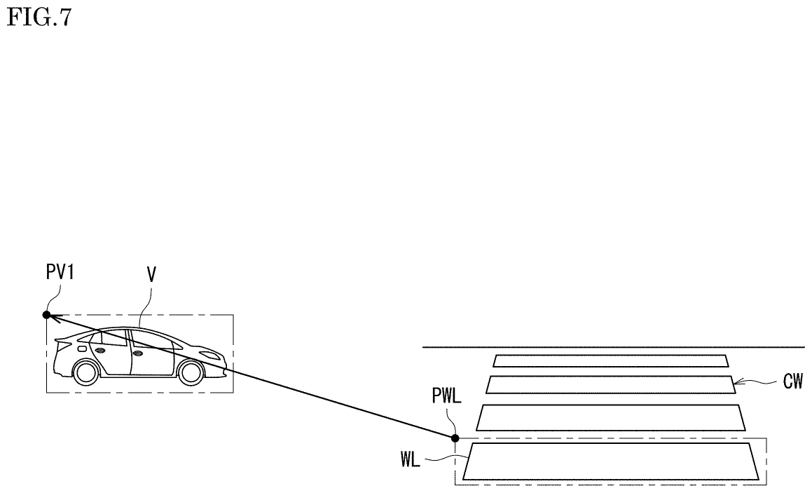

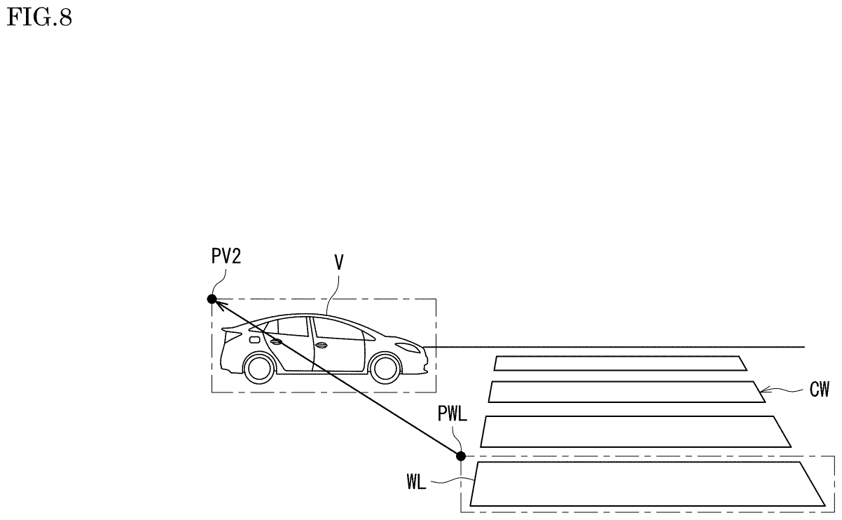

The vehicle velocity detecting processing is specifically described referring to FIGS. 7 and 8. FIG. 7 is an image taken at the time t−1, and FIG. 8 is an image taken at the time t. First, on the image taken at the time t−1 (FIG. 7), a boundary box is set with respect to the white line WL located at the closest position to the user, and also a boundary box is set with respect to the vehicle V. The processing is performed using the training data for recognizing the white line WL by deep learning and the training data for recognizing the vehicle V by deep learning, in the same way as described above. The reference coordinate points PWL and PV1 are set with respect to the respective boundary boxes (in FIG. 7, the positions of the left top corners of the respective boundary boxes). Also, on the image taken at the time t−1 (FIG. 7), a vector from the reference coordinate point PWL of the boundary box set with respect to the white line WL to the reference coordinate point PV1 of the boundary box set with respect to the vehicle V is defined. This vector is referred to as a “first velocity vector”. After that, on the image taken at the time t (FIG. 8), a vector from the reference coordinate point PWL of the boundary box set with respect to the same white line WL to the reference coordinate point PV2 of the boundary box set with respect to the vehicle V (the vehicle that has moved for the period from the time t−1 to the time t) is defined. This vector is referred to as a “second velocity vector”.

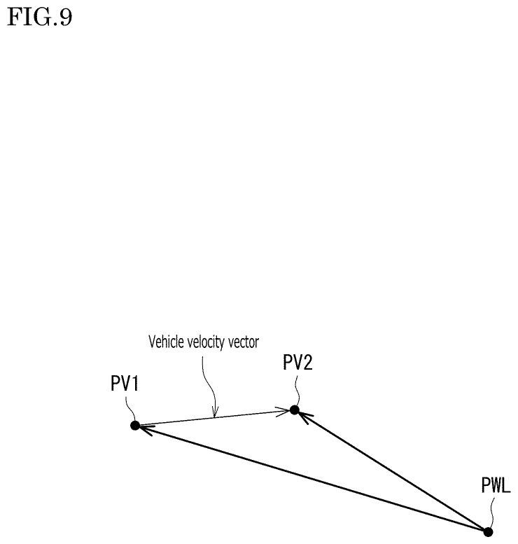

Then as shown in FIG. 9, the difference between the first velocity vector and the second velocity vector is calculated as the vehicle velocity (i.e. the vehicle velocity obtained by the vehicle velocity vector). In this way, even when the respective positions of the white line WL and the vehicle V in the image vary due to swing of the white cane 1 side to side, it is possible to correctly calculate the vehicle velocity.

In the preliminary estimating operation of this embodiment, the threshold values for extracting the vehicle that is estimated to have a possibility of contact are defined as v1 and v2 (v2>v1>0). Also, as the range of the vehicle velocity v, the following are defined: the range of v1>v≥0 (very low); the range of v2>v≥v1 (low); and the range of v≥v2 (intermediate).

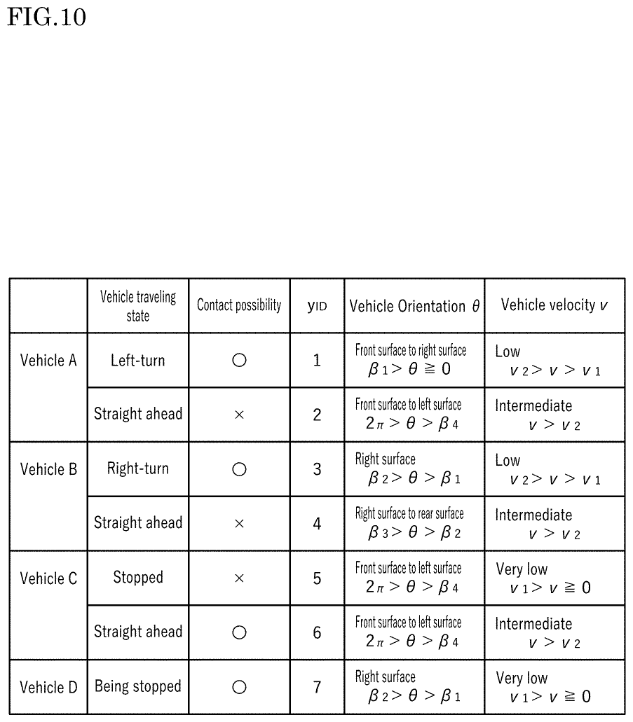

FIG. 10 is a table for explaining the preliminary estimating operation. In the preliminary estimating operation, the vehicles that are estimated to have a possibility of contact are narrowed down by screening according to the traveling state of the vehicle.

As explained referring to FIG. 6, in the state in which four vehicles A to D are travelling or are stopped in the vicinity of the intersection, it is possible to recognize, as described above, the traveling state (the vehicle orientation and the vehicle velocity) of each of the respective vehicles A to D based on the information on the images transmitted from the camera 20 at predetermined intervals.

Also as shown in FIG. 6, when the vehicle A enters the intersection from the left and diagonally forward direction viewed from the user U, the traveling direction of the vehicle A that enters the intersection is estimated to be the straight ahead direction or the left-turn direction, as described above. In this case, when the vehicle A travels straight ahead, it is estimated that there is no possibility of contact with the user U. However, when the vehicle A turns left and the vehicle velocity is low, it is estimated that there is a possibility of contact with the user U.

In the table in FIG. 10, the reference sign “x” in the column of the “contact possibility” means that there is no possibility of contact with the user U. The reference sign “o” means that there is a possibility of contact with the user U. That is, when the vehicle A that has entered the intersection from the left and diagonally forward direction viewed from the user U turns left, the orientation of the vehicle A with respect to the camera 20, which is referred to as “θ”, transits from the front surface to the right front surface. In this process, the surface of the vehicle A that faces the camera 20 falls into the vehicle orientation threshold value range of 0° to β1 (β1>1≥0). Also, since the vehicle A slows down for turning left, the vehicle velocity v becomes low. In this process, the vehicle velocity falls into the low range (v2>v≥v1). On these conditions, when the moving direction of the vehicle A is left-turn direction and furthermore the vehicle velocity v is low, it is estimated that there is a possibility of contact with the user U.

Specifically, this estimation is obtained by the expression (1) below where the vehicle orientation θ and the vehicle velocity v are variables.

[Mathematical 1]

y_{ID}=ψ₁(θ,v) (1)

Here, y_{ID} represents the judgment value (judgment value obtained by calculation) for estimating whether there is a possibility of contact of the vehicle with the user, θ represents the vehicle orientation defined according to the vehicle orientation threshold value, and v represents the vehicle velocity (the range of the vehicle velocity). In this embodiment, when y_{ID} is obtained as any of “1, 3, 6 and 7” (i.e. y_{ID}∈{1, 3, 6, 7}), the vehicle is estimated to have a possibility of contact with the user, while y_{ID} is obtained as any of “2, 4 and 5” (i.e. y_{ID}∈{2, 4, 5}), the vehicle is estimated to have no possibility of contact with the user. When the vehicle A turns left, the expression y_{ID}=1 is obtained. Thus, the vehicle A is estimated to have a possibility of contact with the user.

Also, when the vehicle B enters the intersection from the left and diagonally backward direction viewed from the user U, the traveling direction of the vehicle B that enters the intersection is estimated to be the straight ahead direction or the right-turn direction, as described above. In this case, when the vehicle B travels straight ahead, it is estimated that there is no possibility of contact with the user U (the reference sign is “x” in the column of “contact possibility”). However, when the vehicle B turns right and the vehicle velocity is low, it is estimated that there is a possibility of contact with the user U (the reference sign is “o” in the column of “contact possibility”).

That is, when the vehicle B that has entered the intersection from the left and diagonally backward direction viewed from the user U turns right, the orientation θ of the vehicle B with respect to the camera 20 transits from the right side surface to the right rear surface. In this process, the surface of the vehicle B that faces the camera 20 falls into the vehicle orientation threshold value range of β2 to β1 (β2>θ>β1). Also, since the vehicle B slows down for turning right, the vehicle velocity v becomes low. In this process, the vehicle velocity falls into the low range (v2>v≥v1). On these conditions, when the moving direction of the vehicle B is right-turn direction and furthermore the vehicle velocity v is low, it is estimated that there is a possibility of contact with the user U.

When the vehicle C enters the intersection from the right direction viewed from the user U, the traveling direction of the vehicle C is estimated to be the straight ahead direction, or the vehicle C is estimated to be stopped, as described above. When the vehicle C is stopped, it is estimated that there is no possibility of contact with the user U (the reference sign is “x” in the column of “contact possibility”). However, when the vehicle C travels straight ahead and the vehicle velocity is intermediate, it is estimated that there is a possibility of contact with the user U (the reference sign is “o” in the column of “contact possibility”).

That is, when the vehicle C that has entered the intersection from the right direction viewed from the user U travels straight ahead, the orientation θ of the vehicle C with respect to the camera 20 is in the range of the front surface to the left front surface. That is, the surface of the vehicle C that faces the camera 20 falls into the vehicle orientation threshold value range of 2π to β4 (2π>θ>β4). Also, since the vehicle C does not slow down, the vehicle velocity v falls into the intermediate range (v≥v2). On these conditions, when the moving direction of the vehicle C is straight ahead direction and furthermore the vehicle velocity v is intermediate, it is estimated that there is a possibility of contact with the user U.

When the vehicle D is being stopped at a point in the right and diagonally forward direction viewed from the user U, it is estimated that there is a possibility of contact with the walking user U (the reference sign is “o” in the column of “contact possibility”). That is, when the vehicle D is being stopped at a point in the right and diagonally forward direction viewed from the user U, the orientation of the vehicle D with respect to the camera 20 is in the range of the right rear surface. Thus, the surface of the vehicle D that faces the camera 20 falls into the vehicle orientation threshold value range of β2 to β1 (β2>θ>β1). Also, since the vehicle D is being stopped, the vehicle velocity v falls into the range (v1>v≥0). On these conditions, the vehicle D is estimated to have a possibility of contact with the user U.

The above-described conditions of the range of the vehicle velocity based on which it is estimated that there is a possibility of contact with the user U correspond to the feature that “as to the extraction condition of the moving body estimated to have the possibility of contact, the range of a moving velocity condition for estimating that the moving body has the possibility of contact when the moving body approaches the movement support apparatus without changing the moving direction is set higher than the range of the moving velocity condition for estimating that the moving body has the possibility of contact when the moving body approaches the movement support apparatus with changing the moving direction” in the present invention.

In this way, only the vehicle that is estimated to have a possibility of contact is extracted, among the plurality of vehicles A to D, by screening according to the traveling state of the vehicle. For example, when the vehicle A turns left, the vehicle B travels straight ahead, the vehicle C is stopped and the vehicle D starts moving, only the vehicle A among the four vehicles A to D is estimated to have a possibility of contact with the user U, accordingly, the vehicle A is solely extracted. Also in another case where the vehicle A travels straight ahead, the vehicle B travels straight ahead, the vehicle C is stopped and the vehicle D remains in the stopped state, only the vehicle D among the four vehicles A to D is estimated to have a possibility of contact with the user U, accordingly, the vehicle D is solely extracted.

The preliminary estimating operation was thus described.

### (Contact Estimating Operation)

The contact estimating operation performed by the contact estimating part 87b is to determine whether there is a possibility of contact of the user with the vehicle extracted by the preliminary estimating operation while there is a distance between the user and the vehicle.

More specifically, the contact estimating part 87b determines whether there is a possibility of contact of the user with the vehicle based on the information (contact determination support information) such as the relative distance between a fixed object (in this embodiment, the white line WL of the crosswalk CW) that is located toward the front of the user in the walking direction and the vehicle (the vehicle extracted by the preliminary estimating operation), the vehicle velocity, and the relative distance between the vehicle and the white cane 1.

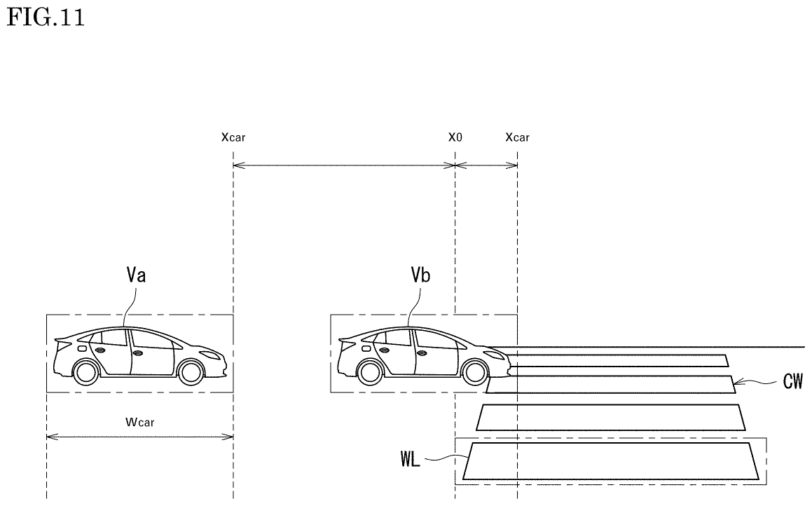

Referring to FIG. 11 (diagram for explaining the contact estimating operation), two positions of the vehicle are considered as the traveling position of the vehicle. The position of a vehicle Va in FIG. 11 is right before the crosswalk CW (on the left side of FIG. 11), and the position of a vehicle Vb in FIG. 11 is on the crosswalk CW (more specifically, the position where the front wheels of the vehicle reach the crosswalk CW).

In FIG. 11, the right end position of the boundary box set with respect to the vehicle is defined as X_{car}, which is a coordinate position in the horizontal direction in the image (i.e. the position on the coordinate on which the value increases toward the right direction), and the left end position of the boundary box set with respect to the white line WL located at the frontmost of the crosswalk CW is defined as X₀. Therefore, when the vehicle is at the position of the vehicle Va, the value of X₀-X_{car} is a positive value while the vehicle is at the position of the vehicle Vb, the value of X₀-X_{car} is a negative value. Also, when the front end position of the vehicle coincides with the left end position of the boundary box of the white line WL located at the frontmost of the crosswalk CW, the value of X₀-X_{car} is 0.

In view of the above, in this embodiment, when the expression (2) below holds (i.e. when g_{c}<0 holds) in the contact estimating operation, it is estimated that there is a possibility of contact with the vehicle. When the expression (2) does not hold (i.e. when g_{c}≥0 holds), it is estimated that there is no possibility of contact with the vehicle.

[Mathematical 2]

g_{c}=x₀−x_{car}+δ₁(v)+δ₂(w_{car})<0 (2)

Here, δ₁ (v) is a correction term according to the vehicle velocity, and δ₂(w_{car}) is a correction term according to the relative distance between the vehicle and the white cane 1 (in other words, the relative distance between the vehicle and the user), which is a correction term according to the relative distance obtained by the vehicle length (length in the front-back direction of the vehicle body) w_{car} in the image. In this case also, since the vehicle length w_{car} varies according to various kinds of vehicles, it is preferable that data corresponding to such various kinds of vehicles (data on the length w_{car}) isannotated in advance.

Here, the respective correction terms are described. In the case where the vehicle velocity is high, even when the vehicle has not yet reached the crosswalk at the current time (see, for example, the position of the vehicle Va), the vehicle Va may reach the crosswalk CW within a relatively short time. Taking into account the above, in the expression (2), the correction term δ₁ (v) according to the vehicle velocity is added (i.e. δ₁ (v) is added, which increases the absolute value of its negative value as the vehicle velocity increases), so that g_{c} is obtained as a smaller value as the vehicle velocity becomes higher. Thus, the accuracy of the determination is improved in consideration of the vehicle velocity.

Also, in the case where the relative distance between the vehicle and the white cane 1 is small, even when the vehicle has not yet reached in front of the user in the walking direction at the current time, after that the vehicle may make contact with the user because the movement of the vehicle and the walk of the user continue. Taking into account the above, in the expression (2), the correction term δ₂(w_{car}) according to the relative distance between the vehicle and the white cane 1 is added (i.e. δ₂(w_{car}) is added, which increases the absolute value of its negative value as the relative distance between the vehicle and the white cane 1 becomes smaller), so that g_{c} is obtained as a smaller value as the relative distance between the vehicle and the white cane 1 becomes smaller. Thus, the accuracy of the determination is improved in consideration of the relative distance.

The contact estimating operation was thus described. When it is determined, by the contact estimating operation, that there is a possibility of contact of the vehicle with the white cane 1 (user), the information transmitting section 88 outputs instruction information on the movement support operation for performing the movement support operation to the vibration generator 50 so that the vibration generator 50 vibrates in a pattern indicating the stop instruction (stop notification).

### —Walking Support Operation—

Next, the walking support operation (movement support operation) by the movement support device configured as described above is described. To begin with, the outlines of the walking support operation is described.

### (Outlines of Walking Support Operation)

Here, the time when the user is walking is represented by t∈[0, T], and the variable indicating the state of the user (state variable) is represented by s∈R^{T}. The state variable at the time t is indicated by an integer falling into sₜ∈{−1, 0, 1, 2}. Specifically, the system stopped state is indicated by sₜ=−1, the walking state is indicated by sₜ=0, the stopping state is indicated by sₜ=1, and the crossing state is indicated by sₜ=2. The system stopped state here means the state in which the movement support device 10 is stopped because the stop condition of the system is met. Specifically, in the movement support device 10 of this embodiment, if the state in which the crosswalk detecting section 82 does not recognize the crosswalk CW continues for a predetermined period of time (i.e. if the stop condition of the system is met), the movement support device 10 is stopped. Thus, the system stopped state means the state in which the movement support device 10 is stopped due to satisfaction of the stop condition of the system. The walking state is assumed, for example, to be the state in which the pedestrian is walking toward an intersection (intersection with the traffic light TL and the crosswalk CW). Also, the stopping state is assumed to be the state in which the user has reached in front of the crosswalk CW and is stopping and waiting for the traffic light to change (i.e. waiting for change of the traffic light from red light to green light). That is, it is the state in which the user is not walking. Also, the crossing state is assumed to be the state in which the user is walking across the crosswalk CW.

In this embodiment, an algorithm is proposed, which is to obtain the output (output variable) y∈R^{T} to support the user's walking when an image Xt∈R^{w0×h0} (w₀ and h₀ respectively indicate the height and width of the image) taken by the camera at the time t is input. Here, the output to support the user's walking is indicated by an integer falling into yₜ∈{1, 2, 3, 4, 5}. Specifically, the stop instruction is indicated by yₜ=1, the walk instruction is indicated by yₜ=2, the right deviation warning is indicated by yₜ=3, the left deviation warning is indicated by yₜ=4, and the system stop notice is indicated by yₜ=5. In the description below, the stop instruction is occasionally referred to as the “stop notification”. Also, the walk instruction is occasionally referred to as the “walk notification” or the “cross notification”. These instructions (notifications) and warnings are given to the user by the vibrations in certain patterns of the vibration generator 50. The user understands in advance the relationships between the instructions (notifications)/warnings and the vibration patterns of the vibration generator 50. Thus, the user recognizes the kind of instructions/warnings by feeling the certain pattern of vibration of the vibration generator 50 via the handgrip part 3.

Also, there are functions (hereinafter referred to as the “state transition functions”) f₀, f₁, f₂ and f₃ as described in detail later. The functions f₀, f₁ and f₂ are the state transition functions to determine the transition of the variable s indicating the state of the user, and the function f₃ is the state transition function to determine the deviation from the crosswalk (deviation in the left and right direction). These state transition functions f₀ to f₃ are stored in the ROM. Specific examples of the state transition functions f₀ to f₃ are described later.

### (Outlines of Output Variable y and State Transition Function fᵢ)

Here, a description is given on the output yₜ∈{1, 2, 3, 4, 5} to support the user's walking.

As described above, the output yₜ to support the user's walking constitutes five kinds of outputs, that is, the stop instruction (yₜ=1), the walk instruction (yₜ=2), the right deviation warning (yₜ=3), the left deviation warning (yₜ=4), and the system stop notice (yₜ=5).

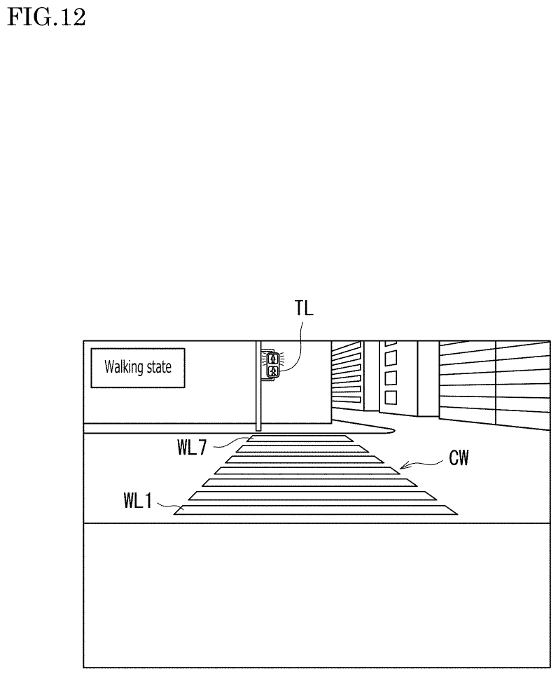

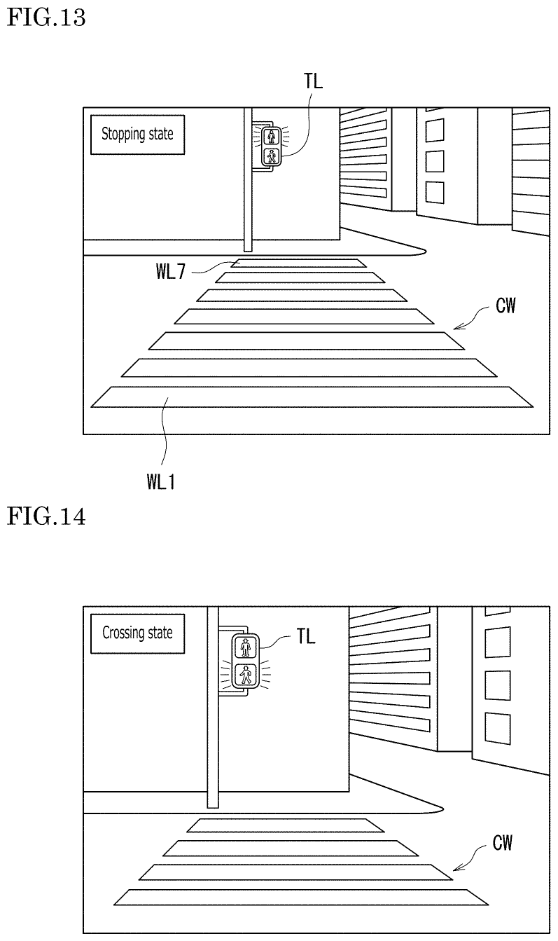

The stop instruction (yₜ=1) is to instruct the user to stop walking at the time when the walking user reaches in front of the crosswalk. For example, in the case where the image taken by the camera 20 shows the state in FIG. 12 (diagram exemplarily illustrating the image taken by the camera 20 when the user is in the walking state toward the crosswalk CW), the distance to the crosswalk CW is relatively long, and thus the stop instruction (yₜ=1) is not output and the user remains in the walking state (sₜ=0). In the case where the image taken by the camera 20 shows the state in FIG. 13 (diagram exemplarily illustrating the image taken by the camera 20 at a timing when the user arrives at the crosswalk CW), it is a timing when the user reaches in front of the crosswalk CW, and thus, the stop instruction (yₜ=1) is output to instruct the user to stop walking. The determination on whether the condition for giving the stop instruction (yₜ=1) is satisfied or not (i.e. determination based on the calculation results of the state transition function) will be described later.

The walk instruction (yₜ=2) is to instruct the user to walk (walk across the crosswalk CW) when the traffic light TL has changed from red to green. For example, when the traffic light TL has changed from red to green in the image taken by the camera 20 while the user is in the stopping state (sₜ=1) in front of the crosswalk CW, the walk instruction (yₜ=2) is output to instruct the user to start crossing the crosswalk CW. The determination on whether the condition for giving the walk instruction (yₜ=2) is satisfied or not (i.e. determination based on the calculation results of the state transition function) will also be described later.

In this embodiment, the timing to give the walk instruction (yₜ=2) is set to the timing when the traffic light TL has changed from red to green. That is, if the traffic light TL has already changed to green when the user arrives at the crosswalk CW, the walk instruction (yₜ=2) is not given, and at the timing when the traffic light TL that had again changed to red has changed to green, the walk instruction (yₜ=2) is given. In this way, it is possible to ensure a sufficient period of time during which the traffic light TL remains green so that the user can cross the crosswalk CW, which prevents the change of the traffic light TL from green to red while the user is still crossing the crosswalk CW.

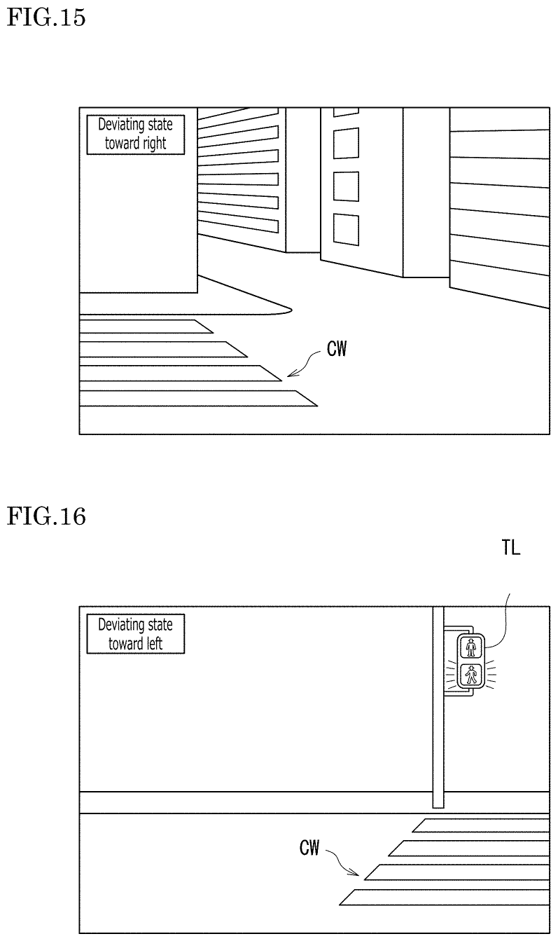

The right deviation warning (yₜ=3) is to warn the user that he/she may deviate from the crosswalk CW toward the right direction when the user who crosses the crosswalk CW is walking in the direction deviating from the crosswalk CW toward the right direction. For example, when the image taken by the camera 20 shows the state in FIG. 14 (diagram exemplarily illustrating the image taken by the camera 20 when the user is in the crossing state on the crosswalk CW) in which the user is in the crossing state (sₜ=2) on the crosswalk CW, and then when the image taken by the camera 20 shows the state in FIG. 15 (diagram exemplarily illustrating the image taken by the camera 20 when the user in the crossing state on the crosswalk CW is walking in a direction deviating from the crosswalk CW toward the right direction), this change shows the fact that the user is walking in the direction deviating from the crosswalk CW toward the right direction. Thus, the right deviation warning (yₜ=3) is output to warn the user.

The left deviation warning (yₜ=4) is to warn the user that he/she may deviate from the crosswalk CW toward the left direction when the user who crosses the crosswalk CW is walking in the direction deviating from the crosswalk CW toward the left direction. For example, when the image taken by the camera 20 shows the state in FIG. 14 in which the user is in the crossing state (sₜ=2) on the crosswalk CW, and then when the image taken by the camera 20 shows the state in FIG. 16 (diagram exemplarily illustrating the image taken by the camera 20 when the user in the crossing state on the crosswalk CW is walking in a direction deviating from the crosswalk CW toward the left direction), this change shows the fact that the user is walking in the direction deviating from the crosswalk CW toward the left direction. Thus, the left deviation warning (yₜ=4) is output to warn the user.

The determination on whether the respective conditions for giving the right deviation warning (yₜ=3) and the left deviation warning (yₜ=4) are satisfied or not (i.e. determination based on the calculation results of the state transition function) will also be described later.

The system stop notice (yₜ=5) is to notify to the user that the movement support device 10 is stopped when the stop condition of the system is satisfied. Specifically, when an obstacle exists on the crosswalk CW and whole of the crosswalk CW is covered by the obstacle in the image acquired by the camera 20 (i.e. all or almost all of the white lines WL1 to WL7 of the crosswalk CW are covered by the obstacle), it is not possible to recognize the existence of the crosswalk CW by the image acquired by the camera 20 (i.e. it is not possible to determine whether the crosswalk CW exists or not). In other words, there is a possibility of giving the stop notification although there is no crosswalk CW (i.e. the stop notification derived from the existence of the obstacle is given), which may affect the sufficient reliability of the operations of the movement support device 10 (reliability of the stop notification). In this situation, since the state in which the existence of the crosswalk CW cannot be recognized continues for a certain period of time, it is used as a condition for stopping the movement support device 10 so as not to output the false stop notification. Furthermore, the information on the stop of the movement support device 10 is transmitted to the vibration generator 50 so that the vibration generator 50 notifies to the pedestrian that the movement support device 10 is stopped.

### (Feature Value to Support Walking)

Here, the feature value used to support the user's walking is described. In order to appropriately give, to the user, the various kinds of notifications such as the stop notification to instruct to stop walking in front of the crosswalk CW and the crossing start notification given after that, it is definitely required to precisely recognize the position of the crosswalk CW (position of the white line WL1 located at the frontmost of the crosswalk CW) and furthermore the state of the traffic light TL (whether it is green or red) based on the information from the camera 20. That is, a model formula is required to be developed by reflecting the position of the white line WL1 and the state of the traffic light TL so as to recognize the current situation that the user is in according to the model formula.

The description on the feature value and the state transition function below is given on the case where no obstacle exists on the crosswalk CW and thus the crosswalk CW is recognized (at least the white line WL1 located at the frontmost of the crosswalk CW is recognized) in the image acquired by the camera 20, as the basic operations of the movement support device 10.

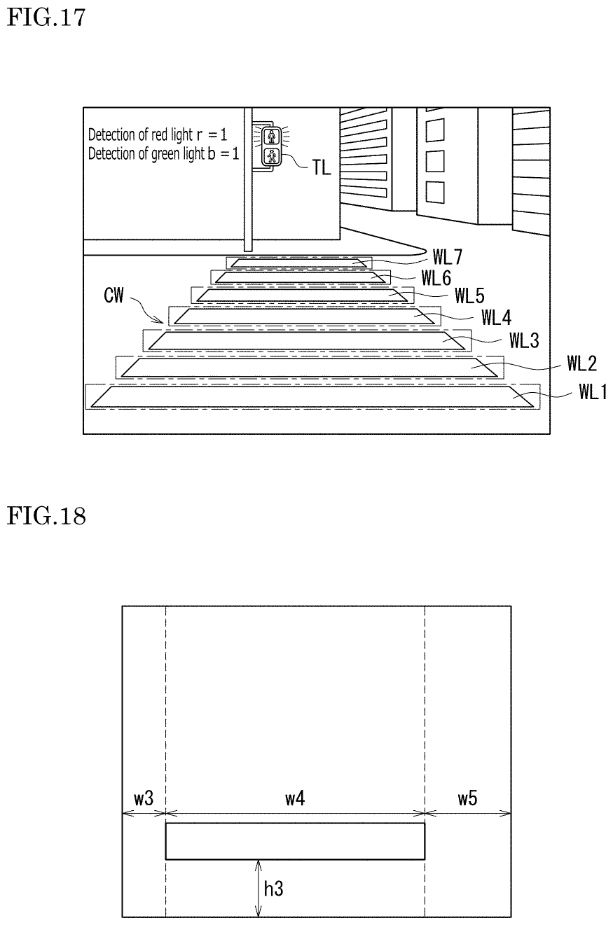

FIGS. 17 and 18 show outlines of the feature values that fit into {w₃, w₄, w₅, h₃, r, b}^{T}∈R⁶, which is used to support the user's walking. Here, r and b respectively represent the detection results (0: undetected; 1: detected) of the state of the traffic light TL (i.e. red or green). When detecting the state of the traffic light TL, an area A1 surrounded by the dashed line is extracted as described above referring to FIG. 4 so as to recognize the state of the traffic light TL. Also, w₃, w₄, w₅ and h₃ are defined as shown in FIG. 18 using the boundary box set with respect to the white line WL1 located at the frontmost among the white lines WL1 to WL7 of the crosswalk CW recognized by the crosswalk detecting section 82. That is, w₃ is a distance from the left end of the image to the left end of the boundary box (corresponding to the left end of the white line WL1), w₄ is a width of the boundary box (corresponding to the width of the white line WL1), w₅ is a distance from the right end of the image to the right end of the boundary box (corresponding to the right end of the white line WL1), and h₃ is a distance from the lower end of the image to the lower end of the boundary box (corresponding to the front end edge of the white line WL1).

When the function for detecting the crosswalk CW and the traffic light TL by deep learning is represented by g, and when the estimated boundary boxes of the crosswalk CW and the traffic light TL using the image Xt∈R^{w0×h0} that is taken by the camera 20 at the time t are expressed by g(Xt), the feature value required to support the user's walking is expressed by the expression (3) below.

[Mathematical 3]

j(t)={w₃(t),w₄(t),w₅(t),h₃(t),r(t),b(t)}^{T}=ϕ○g(Xₜ) (3)

Here, the operator is the following.

[Mathematical 4]

ϕ:R^{p1×4} [symbol] R⁶ (4)

The above operator is to extract the above feature value j(t) by which the above g(Xt) is subjected to post-processing, and p1 is a maximum number of the boundary boxes per 1 frame.

### (State Transition Function)

Here, the state transition function is described. As described above, the state transition function is used to determine whether the respective conditions for giving the stop instruction (yₜ=1), the walk instruction (yₜ=2), the right deviation warning (yₜ=3) and the left deviation warning (yₜ=4) are satisfied or not.

The state amount (state variable) sₜ₊₁ at the time t+1 is expressed by the expression (5) below using the time history information J={j(0), j(1), ( . . . ), j(t)} with respect to the feature value of the crosswalk CW, the current state amount (state variable) sₜ, and the image Xₜ₊₁ taken at the time t+1.

[Mathematical 5]

sₜ₊₁=f(J,sₜ,Xᵢ₊₁) (5)

The state transition function f in the above expression (5) is defined as the expression (6) below according to the state amount at the current time.

$$\begin{matrix}[ Mathematical 6 ] & \\ f ( J , {s}_{t} , {X}_{t + 1} ) = \{ \begin{matrix}{f}_{0} ( J , {X}_{t + 1} ) & {ifs}_{t} = 0 ( walking ) \\ {f}_{1} ( J , {X}_{t + 1} ) & {ifs}_{t} = 1 ( stop walking ) \\ {f}_{2} ( J , {X}_{t + 1} ) & {ifs}_{t} = 2 ( crossing )\end{matrix} & ( 6 )\end{matrix}$$

That is, as the transition of the user's walking, the following is repeated: walking (for example, walking toward the crosswalk CW)→stop walking (for example, stop in front of the crosswalk CW)→crossing (for example, crossing the crosswalk CW)→walking (for example, walking after finishing crossing the crosswalk CW). The state transition function for determining whether the condition for giving the stop instruction (yₜ=1) to the user in the walking state (sₜ=0) is satisfied or not is indicated by f₀ (J, Xₜ₊₁). The state transition function for determining whether the condition for giving the cross (walk) instruction (yₜ=2) to the user in the stopping state (sₜ=1) is satisfied or not is indicated by f₁ (J, Xₜ₊₁). The state transition function for determining whether the condition for giving the walk notification (completion of crossing) to the user in the crossing state (sₜ=2) is satisfied or not is indicated by f₂ (J, Xₜ₊₁). The state transition function for determining whether the condition for giving the warning of the deviation from the crosswalk CW to the user in the crossing state (sₜ=2) is satisfied or not is indicated by f₃ (J, Xₜ₊₁), which is described later.

Hereinafter, the state transition functions according to the respective state amounts (state variables) are specifically described.

### (State Transition Function Applied to Walking State)

The state transition function f₀ (J, Xₜ₊₁) used in the case where the state amount at the current time is the walking state (sₜ=0) is expressed by the expressions (7) to (9) below using the feature value of the above expression (3).

$$\begin{matrix}[ Mathematical 7 ] & \\ {f}_{0} ( J , {X}_{t + 1} ) = H ( {α}_{1} - {h}_{3} ( t + 1 ) ) H ( {w}_{4} ( t + 1 ) - {α}_{2} ) \times δ ( {\sum }_{i = t - t 0}^{t} H ( {α}_{1} - {h}_{3} ( i ) ) H ( {w}_{4} ( i ) - {α}_{2} ) ) & ( 7 )\end{matrix}$$

$$\begin{matrix}[ Mathematical 8 ] & \\ {w}_{4} ( t + 1 ) = {I}_{2}^{T} \{ ϕ ∘ g ( {X}_{t + 1} ) \} & ( 8 )\end{matrix}$$

$$\begin{matrix}[ Mathematical 9 ] & \\ {h}_{3} ( t + 1 ) = {I}_{4}^{T} \{ ϕ ∘ g ( {X}_{t + 1} ) \} & ( 9 )\end{matrix}$$

Here, H is a Heaviside function, and δ is a Delta function. Also, α₁ and α₂ are parameters used as criteria, and t0 is a parameter that specifies the past state to be used. Furthermore, the following equations are satisfied: I₂={0, 1, 0, 0, 0, 0}^{T}, and I₄={0, 0, 0, 1, 0, 0}^{T}.

By the expression (7), the condition of α₁>h₃ and w₄>α₂ was not satisfied at the past time t0, and only when this condition is satisfied for the first time at the time t, “1” is obtained. In the other cases, “0” is obtained. That is, “1” is obtained when it is determined, by satisfaction of α₁>h₃, that the white line WL1 located at the frontmost of the crosswalk CW (i.e. the lower end of the boundary box of the white line) is in front of the user's feet, and furthermore it is determined, by satisfaction of w₄>α₂, that the white line WL1 is extended in the direction orthogonal to the traveling direction of the user (i.e. the width of the boundary box of the white line is larger than the predetermined size).

In this way, when “1” is obtained by the expression (7), it is determined that the condition for giving the stop instruction (yₜ=1) is satisfied, and thus the stop instruction (specifically, instruction/notification to stop walking in front of the crosswalk CW) is given to the user in the walking state.

As the condition on which it is determined that the crosswalk CW exists in front of the user's feet in this embodiment, not only (α₁>h₃) is used, but also (w₄>α₂) is added as the limitation of the width of the detected crosswalk CW. Thus, false detection is prevented in the case where another crosswalk (for example, the crosswalk in the intersection in the direction orthogonal to the traveling direction of the user) other than the crosswalk CW in the traveling direction of the user is included in the image Xₜ₊₁. That is, even when there are a plurality of crosswalks having the different crossing directions from one another in the intersection or the like of the road, it is possible to clearly distinguish the crosswalk CW to be crossed by the user (i.e. the crosswalk CW having the white line WL1 whose width is recognized to be relatively large because the white line WL1 is extended in the direction orthogonal to the direction to be crossed by the user) from the other crosswalks (the crosswalks having the white line whose width is recognized to be relatively narrow). Thus, it is possible to correctly instruct the user to start crossing with high accuracy.

### (State Transition Function Applied to Stopping State)

The state transition function f₁ (J, Xₜ₊₁) used in the case where the state amount at the previous time is the stopping state (sₜ=1) is expressed by the expressions (10) to (12) below.

$$\begin{matrix}[ Mathematical 10 ] & \\ {f}_{1} ( J , {X}_{t + 1} ) = b ( t + 1 ) δ ( {\sum }_{i = t - t 0}^{t} r ( i ) ) & ( 10 )\end{matrix}$$

$$\begin{matrix}[ Mathematical 11 ] & \\ b ( t + 1 ) = {I}_{6}^{T} \{ ϕ ∘ g ( {X}_{t + 1}^{′} ) \} & ( 11 )\end{matrix}$$

$$\begin{matrix}[ Mathematical 12 ] & \\ r ( t + 1 ) = {I}_{5}^{T} \{ ϕ ∘ g ( {X}_{t + 1}^{′} ) \} & ( 12 )\end{matrix}$$

Here, X′ₜ₊₁ is obtained by trimming and enlarging the image Xₜ₊₁. Thus, the X′ₜ₊₁ is an image providing high recognition accuracy of the traffic light TL. Furthermore, the following equations are satisfied: I₅={0, 0, 0, 0, 1, 0}^{T}, and I₆={0, 0, 0, 0, 0, 1}^{T}.

By the expression (10), after that the red light was detected at the past time t0, only when the green light is detected for the first time at the time t, “1” is obtained. In the other cases, “0” is obtained.

In this way, when “1” is obtained by the expression (10), it is determined that the condition for giving the walk (cross) instruction (yₜ=2) is satisfied, and thus the cross instruction (specifically, instruction/notification to cross the crosswalk) is given to the user in the stopping state.

Also, when the crosswalk in the intersection does not have any traffic light, the state transition may not be able to be performed by following the above-described logic. In order to resolve the above problem, a new parameter t1>t0 may be introduced, and when it is determined that the state transition from the stopping state does not occur for the period of time t1, the state may be transited to the walking state.

### (State Transition Function Applied to Crossing State)

The state transition function f₂ (J, Xₜ₊₁) used in the case where the state amount at the previous time is the crossing state (sₜ=2) is expressed by the expression (13) below.

$$\begin{matrix}[ Mathematical 13 ] & \\ {f}_{2} ( J , {X}_{t + 1} ) = δ ( {\sum }_{i = t - t 0}^{t} ( b ( i ) + r ( i ) + H ( {α}_{1} - {h}_{3} ( i ) ) H ( {w}_{4} ( i ) - {α}_{2} ) ) ) & ( 13 )\end{matrix}$$

By the expression (13), only when neither the traffic light nor the crosswalk CW in front of the user's feet is detected for the period of time from the past time t−t0 to the current time t+1, “1” is obtained. In the other cases, “0” is obtained. That is, “1” is obtained only when the user has finished crossing the crosswalk CW and thus neither the traffic light nor the crosswalk CW in front of the user's feet is detected.

In this way, when “1” is obtained by the expression (13), it is determined that the condition for giving the notification of completion of crossing is satisfied, and thus the cross completion notification (specifically, notification of completion of crossing the crosswalk) is given to the user in the walking state.

(State Transition Function to Determine Deviation from Crosswalk)

The state transition function f₃ (J, Xₜ₊₁) used to determine whether the user deviates from the crosswalk CW during crossing the crosswalk CW is expressed by the expressions (14) to (16) below.

$$\begin{matrix}[ Mathematical 14 ] & \\ {f}_{3} ( J , {X}_{t + 1} ) = H ( \frac{max ( {w}_{3} ( t + 1 ) , {w}_{5} ( t + 1 ) )}{{w}_{0}} - {α}_{3} ) & ( 14 )\end{matrix}$$

$$\begin{matrix}[ Matheematical 15 ] & \\ {w}_{3} ( t + 1 ) = {I}_{1}^{T} ( ϕ ∘ g ( {X}_{t + 1} ) \} & ( 15 )\end{matrix}$$

$$\begin{matrix}[ Mathematical 16 ] & \\ {w}_{5} ( t + 1 ) = {I}_{3}^{T} \{ ϕ ∘ g ( {X}_{t + 1} ) \} & ( 16 )\end{matrix}$$

Here, α₃ is a parameter used as criteria. Furthermore, the following equations are satisfied: I₁={1, 0, 0, 0, 0, 0}^{T}, and I₃={0, 0, 1, 0, 0, 0}^{T}.

By the expression (14), when the deviation amount of the position of the detected crosswalk CW from the center of the frame is more than the acceptable amount, “1” is obtained. In the other cases, “0” is obtained. That is, “1” is obtained in the case where the value of w₃ is larger than the predetermined value (in the case of left deviation), and in the case where the value of w₅ is larger than the predetermined value (in the case of right deviation).

In this way, when “1” is obtained by the expression (14), the right deviation warning (yₜ=3) or the left deviation warning (yₜ=4) is given.

### (Walking Support Operation)

Here, the flow of the walking support operation by the movement support device 10 is described.

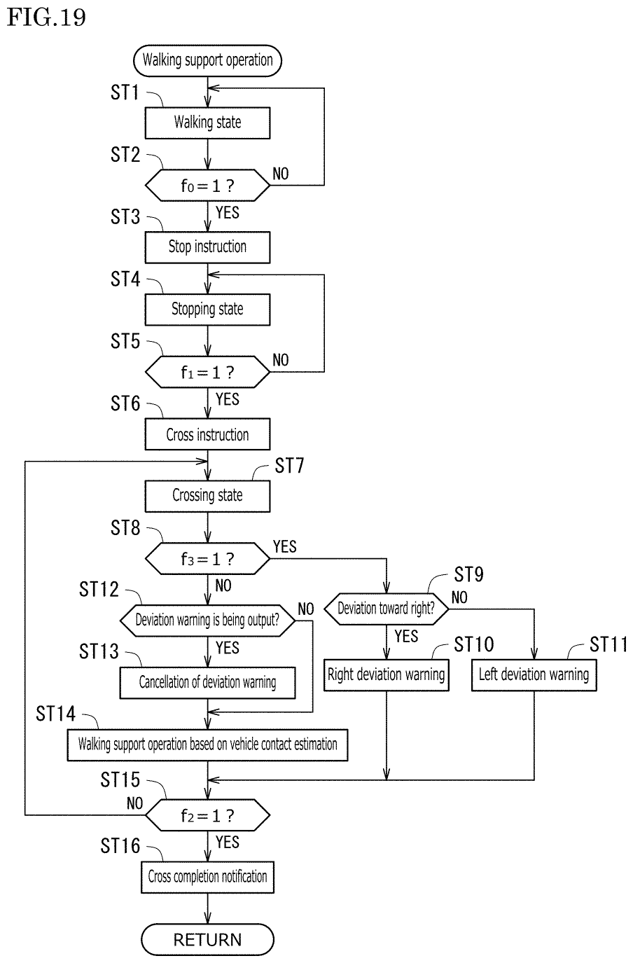

FIG. 19 is a flowchart indicating a series of procedure of the walking support operation described above. The procedure in the flowchart is repeatedly performed at a predetermined time interval such that one routine is executed for the period from the predetermined time t to the predetermined time t+1 in the situation where the user is walking on the road (on the sidewalk). In the description below, the variable (J, Xₜ₊₁) of each state transition function is omitted.

In the situation where the user is in the walking state in step ST1, it is determined, in step ST2, whether “1” is obtained or not by the state transition function f₀ (the above expression (7)) to determine whether the condition for giving the stop instruction (yₜ=1) is satisfied or not based on the position of the white line WL1 of the crosswalk CW in the image area including the crosswalk CW recognized by the crosswalk detecting section 82 (more specifically, the position of the boundary box of the white line WL1 located at the frontmost).

In the case where “0” is obtained by the state transition function f₀, it is determined that the condition for giving the stop instruction (yₜ=1) is not satisfied, i.e. the user has not yet reached in front of the crosswalk CW. Thus, it is determined to be “NO”, and the procedure returns to step ST1. Since it is determined to be “NO” in step ST2 until the user reaches in front of the crosswalk CW, the processes of steps ST1 and ST2 are repeated.

When the user reaches in front of the crosswalk CW and “1” is obtained by the state transition function f₀, it is determined to be “YES” in step ST2. Thus, the procedure advances to step ST3. In step ST3, the stop instruction (yₜ=1) is given to the user. Specifically, the vibration generator 50 in the white cane 1 held by the user vibrates in the pattern indicating the stop instruction (stop notification). Thus, the user who grasps the handgrip part 3 of the white cane 1 feels and recognizes the vibration pattern indicating the stop instruction from the vibration generator 50, and stops walking.

In the situation where the user is in the stopping state in step ST4, it is determined, in step ST5, whether “1” is obtained or not by the state transition function f₁ (the above expression (10)) to determine whether the condition for giving the walk instruction (yₜ=2) is satisfied or not. In the determination processing by the state transition function f₁, the area A1 surrounded by the dashed line is extracted as shown in FIG. 4 described above, and this area A1 is, for example, subjected to enlarging processing. Thus, the state of the traffic light TL can be easily determined.

In the case where “0” is obtained by the state transition function f₁, it is determined that the condition for giving the walk instruction (yₜ=2) is not satisfied, i.e. the traffic light TL has not yet changed to green. Thus, it is determined to be “NO”, and the procedure returns to step ST4. Since it is determined to be “NO” in step ST5 until the traffic light TL changes to green, the processes of steps ST4 and ST5 are repeated.

When the traffic light TL changes to green and “1” is obtained by the state transition function f₁, it is determined to be “YES” in step ST5. Thus, the procedure advances to step ST6. This processing corresponds to the operation by the light-change recognizing section 84 (i.e. light-change recognizing section to recognize that the state of the traffic light changes from the stop instruction state to the crossing permission state).

In step ST6, the walk (cross) instruction (yₜ=2) is given to the user. Specifically, the vibration generator 50 in the white cane 1 held by the user vibrates in the pattern indicating the walk instruction (crossing start notification). Thus, the user who grasps the handgrip part 3 of the white cane 1 recognizes that the walk instruction is given, and starts crossing the crosswalk CW.

In the situation where the user is in the crossing state on the crosswalk CW in step ST7, it is determined, in step ST8, whether “1” is obtained or not by the state transition function f₃ (the above expression (14)) to determine whether the condition for giving the deviation warning from the crosswalk CW is satisfied or not.

When “1” is obtained by the state transition function f₃ and thus it is determined to be “YES” in step ST8, it is determined, in step ST9, whether the deviation from the crosswalk CW is toward the right direction (right deviation) or not. When the deviation direction from the crosswalk CW is the right direction and thus it is determined to be “YES” in step ST9, the procedure advances to step ST10 where the right deviation warning (yₜ=3) is given to the user. Specifically, the vibration generator 50 in the white cane 1 held by the user vibrates in the pattern indicating the right deviation warning. Thus, the user who grasps the handgrip part 3 of the white cane 1 recognizes that the right deviation warning is given, and changes the walking direction to the left direction.

On the other hand, when the deviation direction from the crosswalk CW is the left direction and thus it is determined to be “NO” in step ST9, the procedure advances to step ST11 where the left deviation warning (yₜ=4) is given to the user. Specifically, the vibration generator 50 in the white cane 1 held by the user vibrates in the pattern indicating the left deviation warning. Thus, the user who grasps the handgrip part 3 of the white cane 1 recognizes that the left deviation warning is given, and changes the walking direction to the right direction. After the deviation warning is given as described above, the procedure advances to step ST15.

When the user does not deviate from the crosswalk CW and “0” is obtained by the state transition function f₃, it is determined to be “NO” in step ST8. Thus, the procedure advances to step ST12. In step ST12, it is determined whether the deviation warning is currently being output or not in step ST 10 or step ST11. When the deviation warning is not being output and thus it is determined to be “NO” in step ST12, the procedure advances to step ST14 where the walking support operation is performed based on the vehicle contact estimation (described later).

On the other hand, when the deviation warning is being output and it is determined to be “YES” in step ST12, the procedure advances to step ST13 where the deviation warning is lifted. Then, the procedure advances to step ST14.

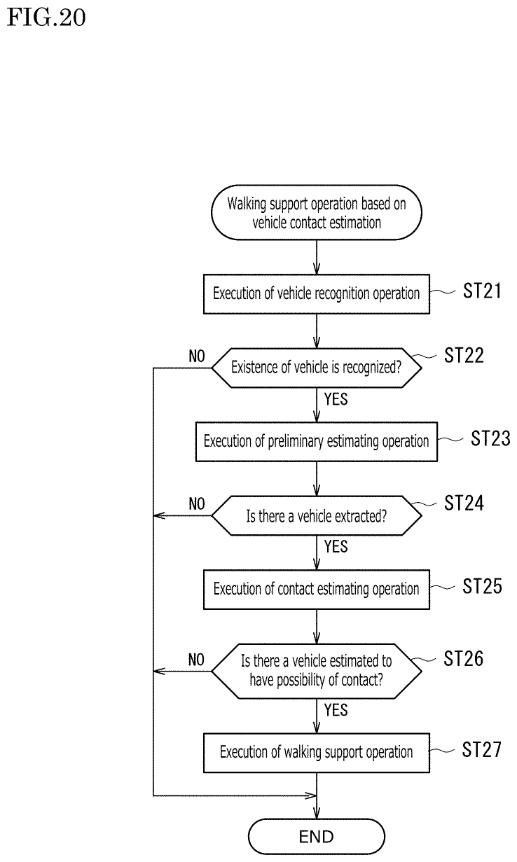

Here, the walking support operation based on the vehicle contact estimation is described referring to FIG. 20. In the walking support operation based on the vehicle contact estimation, the vehicle recognition operation is executed first in step ST21. This is an operation performed by the moving body recognizing section 85 as described above so as to recognize the existence of the vehicle in the image of the information (information on the image taken by the camera 20) received by the information receiving section 81. That is, the vehicle is recognized using the learned model.

Then, the procedure advances to step ST22 where it is determined whether the existence of the vehicle is recognized or not in the image by the vehicle recognition operation. When the existence of the vehicle is not recognized, the procedure exits this subroutine to advance to step ST15 (see FIG. 19). On the other hand, when the existence of the vehicle is recognized, it is determined to be “YES” in step ST22, and the procedure advances to step ST 23 where the preliminary estimating operation is executed by the preliminary estimating part 87a as described above. Specifically, only the vehicle that is estimated to have a possibility of contact is extracted among the recognized vehicles (including the case where one vehicle is recognized) based on information on the relative position relationship with the vehicle and information on changes of the relative position relationship.

After that, the procedure advances to step ST24 where it is determined whether there is a vehicle extracted by the preliminary estimating operation or not. When there is no extracted vehicle, the procedure exits this subroutine to advance to step ST15 (see FIG. 19). On the other hand, when there is an extracted vehicle, it is determined to be “YES” in step ST24. Thus, the procedure advances to step ST25 where the contact estimating operation is executed by the contact estimating part 87b as described above. That is, it is determined whether there is a possibility of contact with the vehicle extracted by the preliminary estimating operation while there is a distance from the vehicle.

After that, the procedure advances to step ST26 where it is determined whether there is a vehicle estimated to have a possibility of contact or not by the contact estimating operation. When there is no vehicle estimated to have a possibility of contact, the procedure exits this subroutine to advance to step ST15 (see FIG. 19). On the other hand, when there is a vehicle estimated to have a possibility of contact, the procedure advances to step ST27 where the walking support operation is executed. The stop instruction (yₜ=1) given to the user in the same way as that in step ST3 is an example of the walking support operation in this case. Specifically, the vibration generator 50 in the white cane 1 held by the user vibrates in the pattern indicating the stop instruction (stop notification). Thus, the user who grasps the handgrip part 3 of the white cane 1 feels and recognizes the vibration pattern indicating the stop instruction from the vibration generator 50, and stops walking. In this way, when there may be a possibility of contact with the vehicle if the user continues to walk, the contact with the vehicle is avoided by instructing the user to stop. After the execution of the walking support operation, the procedure exits this subroutine to advance to step ST15 (see FIG. 19).

Referring to FIG. 19 again, in step ST15, it is determined whether “1” is obtained or not by the state transition function f₂ (the expression (13)) to determine whether the condition for giving the cross completion notification is satisfied or not.

In the case where “0” is obtained by the state transition function f₂, it is determined that the condition for giving the cross completion notification is not satisfied, i.e. the user is still crossing the crosswalk CW. Thus, it is determined to be “NO”, and the procedure returns to step ST7. Since it is determined to be “NO” in step ST15 until the user finishes crossing the crosswalk CW, the processes of steps ST7 and ST15 are repeated.

When the user finishes crossing the crosswalk CW and “1” is obtained by the state transition function f₂, it is determined to be “YES” in step ST15. Thus, the procedure advances to step ST16 where the cross completion notification is given to the user. Specifically, the vibration generator 50 in the white cane 1 held by the user vibrates in the pattern indicating the completion of crossing. Thus, the user who grasps the handgrip part 3 of the white cane 1 recognizes that the cross completion notification is given, and returns to the normal walking.

In this way, the above series of procedure is repeatedly performed every time the user crosses the crosswalk CW.

### —Effects of Embodiment—

In this embodiment as described above, when it is determined that there is a possibility of contact of the vehicle with the white cane 1 (user) by the determination operations of the contact determining section 87 (i.e. the preliminary estimating operation performed by the preliminary estimating part 87a and the contact estimating operation performed by the contact estimating part 87b), the walking support operation by vibration of the vibration generator 50 is started. Thus, it is possible to early recognize the possibility of contact of the user with the vehicle. Furthermore, when there is a possibility of contact, the walking support operation according to the actual situation can be immediately started. As a result, it is possible to appropriately obtain the start timing of the walking support operation.

Also, in the contact estimating operation by the contact estimating part 87b in this embodiment, it is determined whether there is a possibility of contact of the user with only the vehicle extracted by the preliminary estimating operation by the preliminary estimating part 87a. Thus, it is not necessary to determine the possibility of contact with respect to all the vehicles recognized by the moving body recognizing section 85. In other words, no contact estimating operation is required with respect to the vehicle that does not have any possibility of contact. Therefore, it is possible to reduce the calculation burden of the contact estimating part 87b, which contributes to reduction of time required to determine the possibility of contact with the vehicle.

Also, as to the extraction condition (moving velocity condition) of the vehicle estimated to have a possibility of contact in this embodiment, the range of the moving velocity condition for estimating that the vehicle has a possibility of contact when the vehicle approaches the white cane 1 without changing the moving direction (i.e. traveling straight ahead to approach) is set higher than the range of the moving velocity condition for estimating that the vehicle has a possibility of contact when the vehicle approaches the white cane 1 with changing the moving direction (i.e. with making right-turn or left-turn). Thus, the extraction condition of the vehicle estimated to have a possibility of contact is set in light of the actual state of the vehicle velocity. In this way, it is possible to improve extraction reliability of the vehicle estimated to have a possibility of contact.

Also in the preliminary estimating operation by the preliminary estimating part 87a in this embodiment, the extraction condition of the vehicle estimated to have a possibility of contact includes the case where the vehicle is being stopped at a position toward the front of the user in the walking direction (i.e., the vehicle D in FIG. 6 is extracted as a vehicle having the possibility of contact). Thus, it is possible to early determine that there is a possibility of contact of the user with the vehicle (stopped vehicle) as the user walks forward.

Also in this embodiment, since the movement support device is built in the white cane 1, it is possible to provide a valuable white cane 1.

### —Variation—

Now, a variation will be described. This variation is related to a movement support system in which the existence of a user is recognized by a driver of a vehicle by communications between the movement support device 10 and the vehicle V using an in-vehicle information providing device (for example, a navigation system). Here, differences from the above-described embodiment are mainly described.

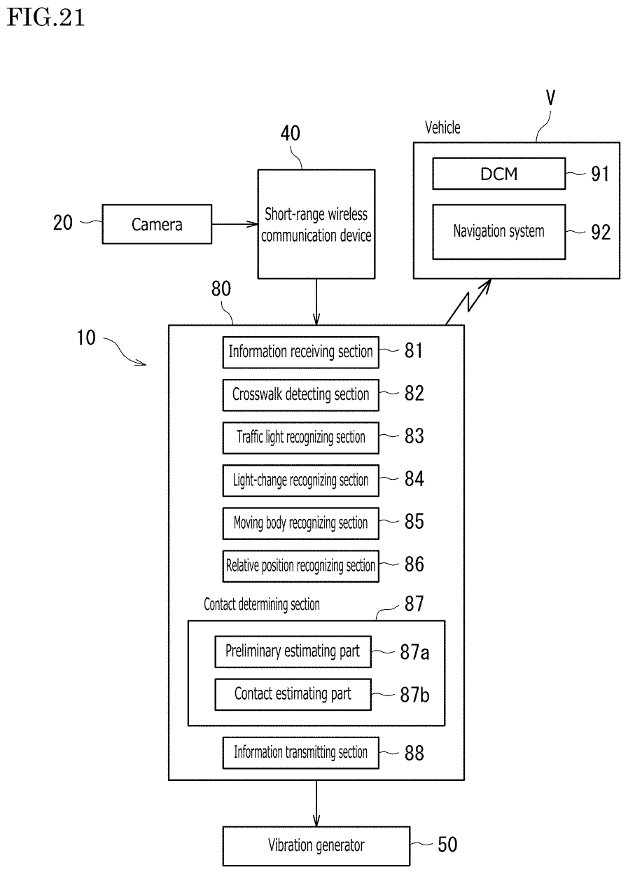

FIG. 21 is a block diagram indicating a schematic configuration of the control system of the movement support system according to this variation.

As shown in FIG. 21, the information transmitting section 88 of the movement support device 10 according to this variation is capable of communicating with a DCM (Data Communication Module) 91 as a wireless communication device (corresponding to the instruction information receiving section in the present invention) mounted on the vehicle V.

The DCM 91 can make bidirectional communication with a navigation system 92 mounted on the vehicle V via an in-vehicle network.

When it is determined that there is a vehicle estimated to have a possibility of contact by the preliminary estimating operation performed by the preliminary estimating part 87a and the contact estimating operation performed by the contact estimating part 87b, the information transmitting section 88 provided in the control device 80 according to this variation identifies the vehicle (specifically, identifies ID information on the vehicle and the like), and outputs the instruction information on the movement support operation to the DCM 91 of the vehicle V. The information transmitting section 88 and the DCM 91 bidirectionally communicate with each other to send and receive the ID information (individual information) on the vehicle V and the instruction information on the movement support operation via predetermined networks including the mobile telephone network and the internet network having many base stations.

The information received by the DCM 91 is transmitted to the navigation system 92, and the voice is emitted from the speaker of the navigation system 92 to notify the driver of the existence of the pedestrian (user) located toward the front of the vehicle (the voice is emitted from the speaker by a control signal from a contact avoidance control section (function section of the CPU) built in the navigation system 92). It is also possible to display the location of the user on the display screen (on the map displayed on the screen) of the navigation system 92.

Thus, by notifying the driver of the existence of the user by the navigation system 92, the driver of the vehicle can notice the existence of the user.

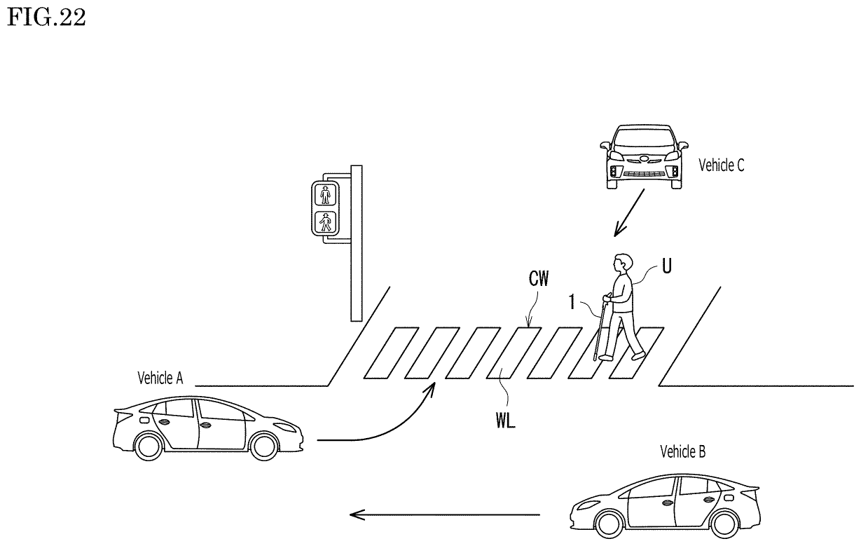

As exemplarily shown in FIG. 22, when the vehicle A turns left, the vehicle B travels straight ahead and the vehicle C is stopped (stopped in front of the crosswalk CW), the instruction information on the movement support operation is output only to the vehicle A among the three vehicles A to C. Thus, the existence of the user U is notified to the driver of the vehicle A by the navigation system 92.

This configuration and/or operations according to this variation may be combined with the above described embodiment but not necessarily required to be combined. Also in this variation, the existence of the user is notified to the driver of the vehicle by the navigation system 92. However, the speaker may be provided in the white cane 1, and the voice may be emitted from the speaker in the white cane 1 to the vehicle so that the driver of the vehicle notices the existence of the user. In this case, it is preferable that a directional speaker is adopted such that the voice is emitted toward the vehicle estimated to have a possibility of contact. Also, an LED light may be provided in the white cane 1, and a light may be emitted from the LED light in the white cane 1 to the vehicle so that the driver of the vehicle notices the existence of the user. In this case, it is preferable that the white cane 1 further includes a drive section to change the irradiation direction such that the light is emitted to the vehicle estimated to have a possibility of contact.

Also, in the case where the vehicle is an autonomous driving vehicle, the vehicle may be emergently stopped when it receives the instruction information on the movement support operation.

### OTHER EMBODIMENTS

The present invention is not limited to the above-described embodiment and the variation. All modifications and changes that come within the meaning and range of equivalency of the claims are intended to be embraced therein.

For example, in the embodiment and the variation as described above, the movement support device 10 is built in the white cane 1 used by the user. However, the present invention is not limited thereto. The movement support device 10 may be built in a cane or a wheel rollator walker for an elderly person as a user. Also, the movement support device 10 may be mounted on a personal mobility.

Also, in the embodiment and the variation as described above, the moving body is a vehicle (automobile). However, the moving body may also include a motorcycle and a bicycle.

Also, in the embodiment and the variation as described above, the charging socket 70 is provided on the white cane 1 so that the battery (secondary battery) 60 is charged from the domestic power supply. However, the present invention is not limited thereto. The battery 60 may be charged by electricity generated by a thin solar sheet adhered onto the surface of the white cane 1. Also, a primary battery may be used in place of the secondary battery. Also, the battery 60 may be charged by a pendulum generator that is built in the white cane 1.

Also, in the embodiment and the variation as described above, the kinds of the notification are classified by the vibration patterns of the vibration generator 50. However, the present invention is not limited thereto. The notification may be given by the voice.

Also, in the preliminary estimating operation in the embodiment and the variation as described above, the vehicle estimated to have a possibility of contact is extracted according to the moving velocity (vehicle velocity) of the vehicle. However, the vehicle estimated to have a possibility of contact may be extracted according to the moving acceleration (vehicle acceleration) of the vehicle. Alternatively, the vehicle estimated to have a possibility of contact may be extracted according to both of the moving velocity and the moving acceleration of the vehicle. Similarly to the case of the determination according to the vehicle velocity, as to the extraction condition of the moving body (vehicle) estimated to have a possibility of contact in this case, “the range of the moving acceleration condition for estimating that the moving body has a possibility of contact when the moving body approaches the movement support apparatus (white cane) without changing the moving direction is set higher than the range of the moving acceleration condition for estimating that the moving body has a possibility of contact when the vehicle approaches the movement support apparatus with changing the moving direction”.

### INDUSTRIAL APPLICABILITY

The present invention is suitably applied to a movement support device that notifies a visually impaired person who is walking of an approaching vehicle.

What is claimed is:

1. A movement support device capable of performing a movement support operation to support movement of a user using a movement support apparatus in which the movement support device is provided, the movement support device comprising:
a moving body recognizing section recognizing a moving body that exists in a vicinity;
a relative position recognizing section recognizing a relative position relationship with the moving body recognized by the moving body recognizing section;
a contact determining section determining whether there is a possibility of contact of the moving body with the movement support apparatus in a state in which there is a distance from the moving body based on contact determination support information including at least one of: information on the relative position relationship with the moving body, which is recognized by the relative position recognizing section; and information on a change of the relative position relationship; and
an information transmitting section outputting instruction information on the movement support operation to perform the movement support operation when the contact determining section determines that there is a possibility of contact of the moving body with the movement support apparatus.

2. The movement support device according to claim 1, wherein
the contact determining section includes: a preliminary estimating part performing a preliminary estimating operation; and a contact estimating part performing a contact estimating operation performed subsequently to the preliminary estimating operation,
in the preliminary estimating operation performed by the preliminary estimating part, when a plurality of moving bodies is recognized by the moving body recognizing section, the preliminary estimating part solely extracts a moving body estimated to have the possibility of contact among the plurality of moving bodies based on the information including at least one kind of; the information on the relative position relationship with each of the plurality of moving bodies; and the information on the change of the relative position relationship, and
in the contact estimating operation performed by the contact estimating part, the contact estimating part determines whether there is a possibility of contact with only the moving body extracted by the preliminary estimating operation based on the contact determination support information in the state in which there is a distance from the extracted moving body.

3. The movement support device according to claim 2, wherein
in the preliminary estimating operation performed by the preliminary estimating part, an extraction condition of the moving body estimated to have the possibility of contact includes a moving direction of the moving body as a direction in which the moving body approaches the movement support apparatus.

4. The movement support device according to claim 3, wherein
in the preliminary estimating operation performed by the preliminary estimating part, the preliminary estimating part extracts the moving body estimated to have the possibility of contact according to a moving velocity of the moving body whose moving direction is the direction in which the moving body approaches the movement support apparatus, and
as to the extraction condition of the moving body estimated to have the possibility of contact, a range of a moving velocity condition for estimating that the moving body has the possibility of contact when the moving body approaches the movement support apparatus without changing the moving direction is set higher than a range of the moving velocity condition for estimating that the moving body has the possibility of contact when the moving body approaches the movement support apparatus with changing the moving direction.

5. The movement support device according to claim 3, wherein
in the preliminary estimating operation performed by the preliminary estimating part, the preliminary estimating part extracts the moving body estimated to have the possibility of contact according to a moving acceleration of the moving body whose moving direction is the direction in which the moving body approaches the movement support apparatus, and
as to the extraction condition of the moving body estimated to have the possibility of contact, a range of a moving acceleration condition for estimating that the moving body has the possibility of contact when the moving body approaches the movement support apparatus without changing the moving direction is set higher than a range of the moving acceleration condition for estimating that the moving body has the possibility of contact when the moving body approaches the movement support apparatus with changing the moving direction.

6. The movement support device according to claim 2, wherein
in the preliminary estimating operation by the preliminary estimating part, an extraction condition of the moving body estimated to have the possibility of contact includes a state in which the moving body is being stopped at a position toward a front of the user in a user's moving direction.

7. The movement support device according to claim 2, wherein
in the contact estimating operation performed by the contact estimating part, the contact determination support information includes a relative distance between a fixed object located toward a front of the user in a user's moving direction and the moving body.

8. The movement support device according to claim 7, wherein
in the contact estimating operation performed by the contact estimating part, the contact determination support information includes a moving velocity of the moving body.

9. The movement support device according to claim 7, wherein
in the contact estimating operation performed by the contact estimating part, the contact determination support information includes a relative distance between the moving body and the movement support apparatus.

10. The movement support device according to claim 1, wherein
the contact determining section performs a determination operation on whether there is a possibility of contact with the moving body on a condition that the user is crossing a road on which the moving body moves.

11. The movement support device according to claim 1 further comprising a notifier for the movement support operation, wherein
the notifier gives a notice to the user by vibration or by voice for supporting the movement of the user.

12. The movement support device according to claim 1, wherein
the user is a visually impaired person, and furthermore the movement support apparatus is a white cane used by the visually impaired person.

13. A movement support system comprising a movement support device capable of performing a movement support operation to support movement of a user using a movement support apparatus in which the movement support device is provided, wherein
the system further includes an instruction information receiving section mounted on a moving body,
the movement support device includes:
a moving body recognizing section recognizing the moving body that exists in a vicinity;
a relative position recognizing section recognizing a relative position relationship with the moving body recognized by the moving body recognizing section;
a contact determining section determining whether there is a possibility of contact of the moving body with the movement support apparatus in a state in which there is a distance from the moving body based on contact determination support information including at least one of: information on the relative position relationship with the moving body, which is recognized by the relative position recognizing section; and information on a change of the relative position relationship; and
an information transmitting section outputting, to the instruction information receiving section of the moving body, instruction information on the movement support operation to perform the movement support operation when the contact determining section determines that there is a possibility of contact of the moving body with the movement support apparatus, and
the moving body includes a contact avoidance control section that performs a contact avoiding operation to avoid the contact with the user when the instruction information receiving section receives the instruction information on the movement support operation.
